(19) United States
(12) Patent Application Publication
(10) Pub. No.: US 2011/0118473 A1
(43) Pub. Date: May 19, 2011

(54) PROCESS FOR CONVERTING 9-DIHYDRO-13ACETYLBACCATIN III PACLITAXEL AND DOCETAXEL

(75) Inventor: Jian Liu, Fredericton (CA)

(73) Assignee: 6570763 Canada Inc., Montreal, QC (CA)

(21) Appl. No.: 12/674,632

(22) PCT Filed: Aug. 21, 2008
(86) PCT No.: PCT/CA08/01490

§ 371 Date: Feb. 22, 2010

Related U.S. Application Data
(60) Provisional application No. 60/935,616, filed on Aug. 22, 2007.
(60) Provisional application No. 61/015,729, filed on Dec. 21, 2007.
(60) Provisional application No. 61/064,561, filed on Mar. 12, 2008.

(51) Int. Cl. C07D 413/12; C07D 305/14

(52) U.S. Cl. 548/215; 549/510

(57) ABSTRACT

Processes for the preparation of docetaxel and paclitaxel or analogs from 9-dihydro-13-acetylbaccatin III via key intermediates (4), (5), (6), (6′), (8) and (8′) or via intermediate (12) as well as processes for the preparation of said intermediates are disclosed. Said processes involve, in particular, oxidation of the hydroxy group at C-10, reaction with side chain precursors and intramolecular isomerisation of the oxo at C-10 to produce the 9-oxo isomer. Paclitaxel and docetaxel are useful in the treatment of cancer. Formulae (I), (II), (III).

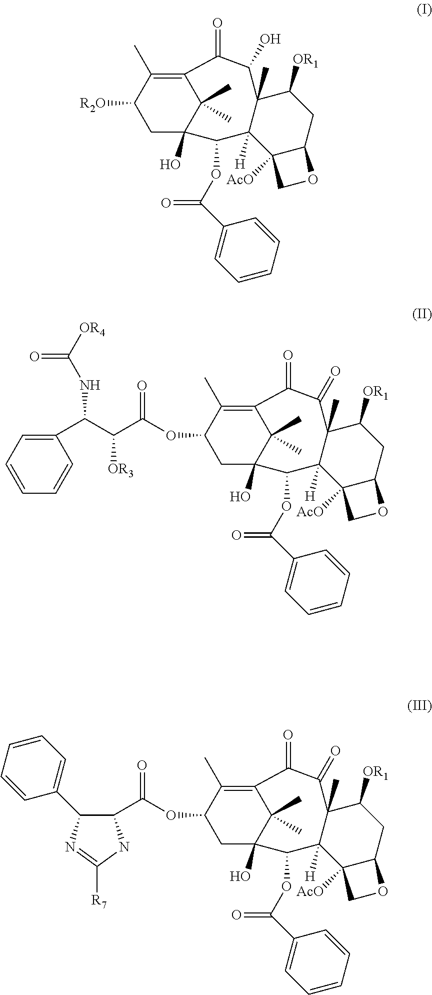

### BACKGROUND OF INVENTION

1. Field of the Invention

The present invention relates to a process for the preparation of docetaxel or paclitaxel, anticancer drugs. More particularly, this invention relates to a process for the synthesis of docetaxel or paclitaxel from 9-dihydro-13-acetylbaccatin III, a taxane compound isolated from Taxus Canadensis, a evergreen bush found in Eastern Canada and Northeastern United States.

2. Brief Description of the Prior Art

Taxanes are substances occurring naturally in yew trees such as Taxus canadensis, which is common in Eastern Canada and the United States. One of the chemicals extracted from the needles of Taxus canadensis is 9-dihydro-13-acetylbaccatin III, which is used to produce, inter alia, 10-deacetylbaccatin III, which is a useful intermediate for the preparation of paclitaxel and analogues thereof.

The taxane family of terpenes is considered to be an exceptionally promising group of cancer chemotherapeutic agents. Many taxane derivatives, including paclitaxel, docetaxel, taxcultine canadensol are highly cytotoxic and possess strong in vivo activities in a number of leukemic and other tumor systems. Paclitaxel, and a number of its derivatives, have been shown to be effective against advanced breast and ovarian cancers in clinical trials. They have also exhibited promising activity against a number of other tumor types in preliminary investigations. Paclitaxel has recently been approved in the U.S. and Canada for the treatment of ovarian cancers.

Due to the structural complexity of docetaxel, partial synthesis is a far more viable approach to providing adequate supplies of docetaxel. Docetaxel was originally invented by Aventis, It went to the market in 1995 and it is a fast growing anticancer drug. This drug is semi-synthetic product, also starting from 10-deacetylbaccatin III. So far the commercial supply of docetaxel comes substantially completely from 10-deacetylbaccatin III. To date, however, the supply of 10-deacetylbaccatin III is limited due to the limited biomass resource and low isolation yield (ranging from 50-165 mg per kilogram of needles of Taxus baccata).

### SUMMARY OF THE INVENTION

It is therefore desirable to provide a process for the preparation of docetaxel or paclitaxel.

It is also desirable to provide a process for the preparation of intermediates useful in the preparation of docetaxel or paclitaxel.

It is also desirable provide processes for the preparation of intermediates useful in the preparation of docatexel or paclitaxel.

### STATEMENTS OF INVENTION

A first broad aspect of the present invention provides new intermediates useful for the preparation of docetaxel and paclitaxel. More particularly, the invention relates to compounds of formula (2), (3), (4), (5), (6), (6′), (8), (8′), (10), (11), (12), (13) and (14):

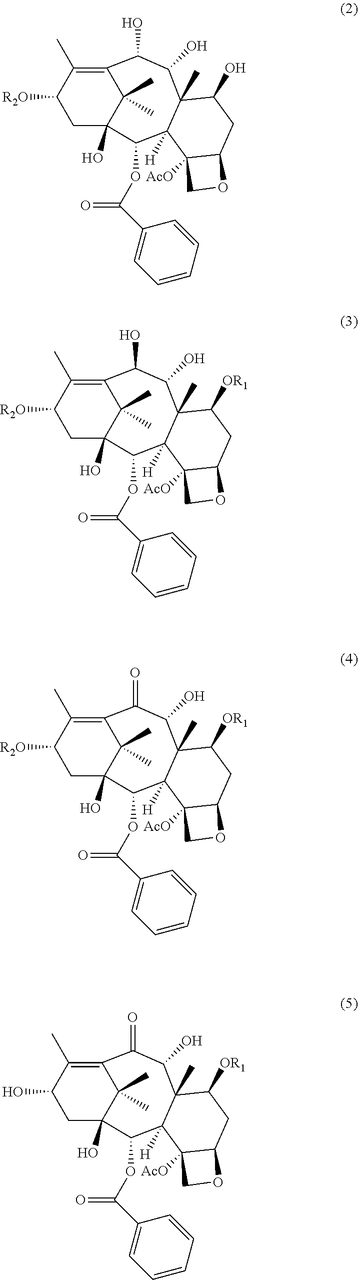

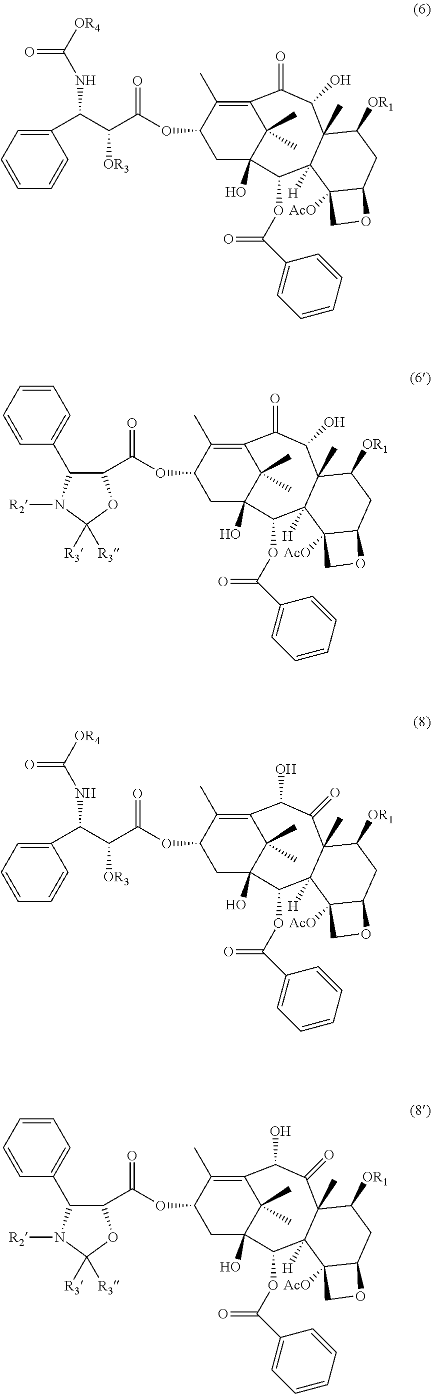

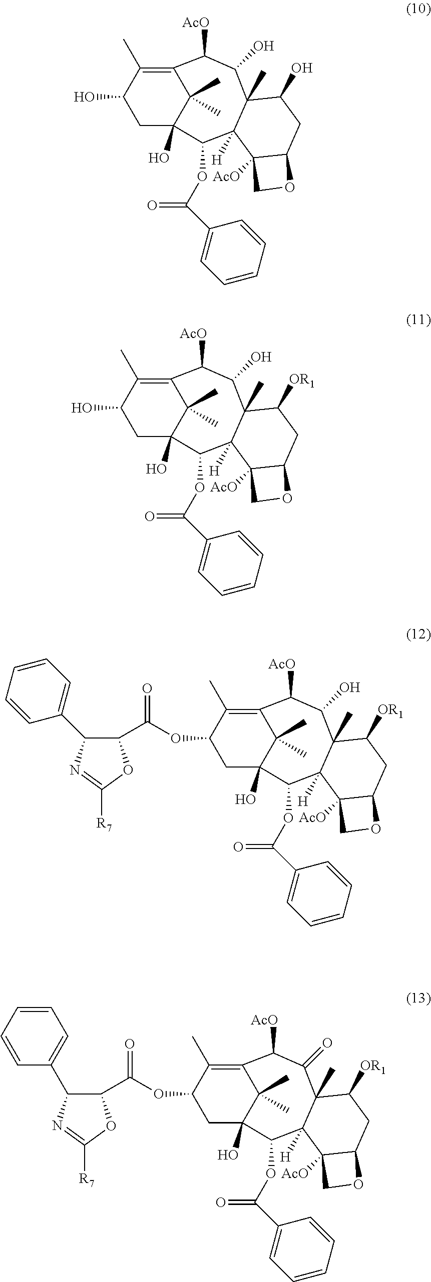

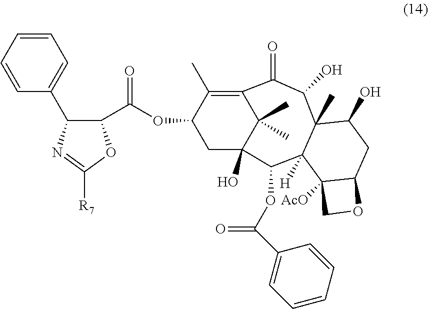

wherein in formula (3), (4), (5), (6), (6′), (8), (8′), (11), (12) and (13), R₁ is a hydrogen atom of a suitable hydroxyl-protecting group;
wherein in formula (2), (3) and (4), R₂ is a hydrogen atom or a suitable hydroxyl-protecting group;
wherein in formula (6′) and (8′), R₂′ is a suitable amino-protecting group and R₃′ and R₃″, identical or different, are a hydrogen atom or a methyl group; or R₂′ and one of R₃′ and R₃″ form together a π-bond and the other of R₃′ and R₃″ is a t-butoxy group or a phenyl group;
wherein in formula (6) and (8), R₃ is a hydrogen atom or a suitable hydroxyl-protecting group;
wherein in formula (6) and (8), R₄ is a hydrogen atom, a linear C₁-C₂₀ alkyl, a branched C₃-C₂₀ alkyl group, a C₁-C₂₀ acyl group, a C₁-C₂₀ halogenated acyl group, a C₃-C₁₂ cycloalkyl, a C₁-C₁₂ heterocyclyl, a C₂-C₂₀ alkenyl, a C₂-C₂₀ alkynyl, a C₆-C₁₂ aryl, a C₆-C₂₀ aralkyl, a C₁-C₂₀ alkyloxy C₆-C₂₀ alkylaryl, a C₁-C₁₂ heteroaryl, a C₂-C₂₀ alkylheterocyclyl or a C₂-C₂₀ alkylheteroaryl,
said alkyl, cycloalkyl, heterocyclyl, alkenyl, alkynyl, aryl, aralkyl, alkylaryl, heteroaryl, alkylheterocyclyl and alkylheteroaryl being unsubstituted or substituted with at least one substituent, each of said substituent(s) being selected from the group consisting of F, Cl, Br, I, OH, SH, NH₂, NO₂, CN, CF₃, —SH, —OCH₂Ph, —OPh, —SCH₃, —SPh, —SCH₂Ph, —COOH, —COOR₆ (in which R₆ is a C₁-C₆ alkyl), linear C₁-C₂₀ alkyl, branched C₃-C₂₀ alkyl, C₆-C₁₂ aryl, C₂-C₂₀ alkenyl, C₁-C₂₀ alkoxy, C₂-C₂₀ alkynyl, C₆-C₂₀ aralkyl, C₆-C₁₂ aryl, C₃-C₈ cycloalkyl, C₁-C₂₀ aminoalkyl, C₆-C₁₂ aminoaryl, C₁-C₁₂ aminoheteroaryl, C₁-C₂₀ hydroxyalkyl, C₆-C₁₂ hydroxyaryl, C₁-C₁₂ hydroxyheteroaryl, C₁-C₁₂ heterocyclyl, C₁-C₁₂ heteroaryl, C₂-C₂₀ alkylheterocyclyl and C₂-C₂₀ alkylheteroaryl; and
wherein in formula (12) and (13), R₇ is C₂-C₁₀ alkynyl, C₁-C₁₀ alkyl, C₂-C₁₀ alkenyl, C₁-C₁₀ alkoxy, C₆-C₁₂ aryl or C₅-C₁₂ heteroaryl, preferably R₇ is a t-butoxy group or a phenyl group
wherein.

A second broad aspect of the present invention preferably provide compounds of formula (2) and (3) which are defined as follows:

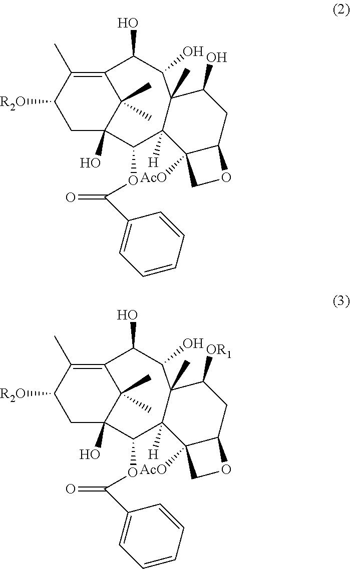

wherein in formula (3) R₁ is a hydrogen atom of a suitable hydroxyl-protecting group; and
wherein R₂ is a hydrogen atom or a suitable hydroxyl-protecting group.

A third broad aspect of the present invention preferably provides compounds of formula (4) or (5) which are defined as follows:

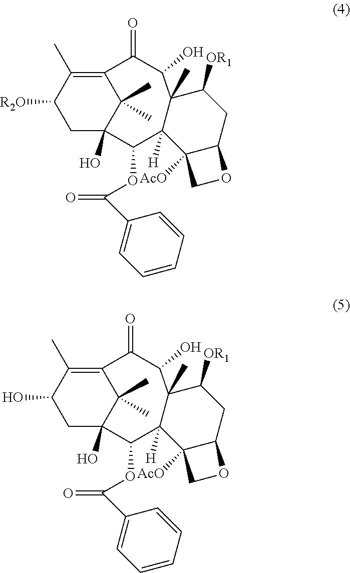

wherein R₁ is a hydrogen atom of a suitable hydroxyl-protecting group; and
wherein in formula (4) R₂ is a hydrogen atom or a suitable hydroxyl-protecting group.

A fourth broad aspect of the present invention preferably provides compounds of formula (6) or (6′) which are defined as follows:

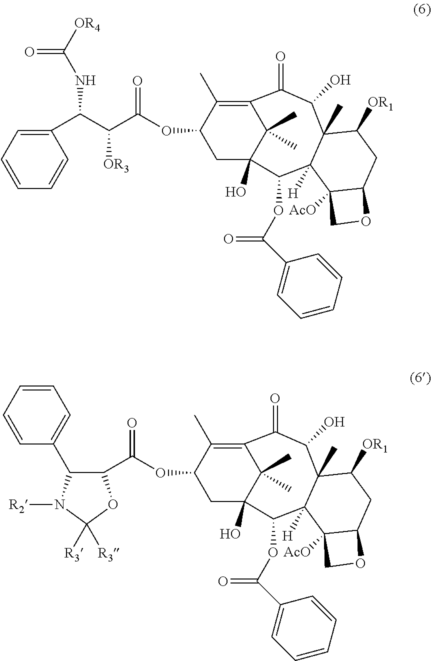

wherein R₁ is a hydrogen atom of a suitable hydroxyl-protecting group;
wherein in formula (6), R₄ is a hydrogen atom, a linear C₁-C₂₀ alkyl, a branched C₃-C₂₀ alkyl group, a C₁-C₂₀ acyl group, a C₁-C₂₀ halogenated acyl group, a C₃-C₁₂ cycloalkyl, a C₁-C₁₂ heterocyclyl, a C₂-C₂₀ alkenyl, a C₂-C₂₀ alkynyl, a C₆-C₁₂ aryl, a C₆-C₂₀ aralkyl, a C₁-C₂₀ alkyloxy C₆-C₂₀ alkylaryl, a C₁-C₁₂ heteroaryl, a C₂-C₂₀ alkylheterocyclyl or a C₂-C₂₀ alkylheteroaryl,
said alkyl, cycloalkyl, heterocyclyl, alkenyl, alkynyl, aryl, aralkyl, alkylaryl, heteroaryl, alkylheterocyclyl and alkylheteroaryl being unsubstituted or substituted with at least one substituent, each of said substituent(s) being selected from the group consisting of F, Cl, Br, I, OH, SH, NH₂, NO₂, CN, CF₃, —SH, —OCH₂Ph, —OPh, —SCH₃, —SPh, —SCH₂Ph, —COOH, —COOR₆ (in which R₆ is a C₁-C₆ alkyl), linear C₁-C₂₀ alkyl, branched C₃-C₂₀ alkyl, C₆-C₁₂ aryl, C₂-C₂₀ alkenyl, C₁-C₂₀ alkoxy, C₂-C₂₀ alkynyl, C₆-C₂₀ aralkyl, C₆-C₁₂ aryl, C₃-C₈ cycloalkyl, C₁-C₂₀ aminoalkyl, C₆-C₁₂ aminoaryl, C₁-C₁₂ aminoheteroaryl, C₁-C₂₀ hydroxyalkyl, C₆-C₁₂ hydroxyaryl, C₁-C₁₂ hydroxyheteroaryl, C₁-C₁₂ heterocyclyl, C₁-C₁₂ heteroaryl, C₂-C₂₀ alkylheterocyclyl and C₂-C₂₀ alkylheteroaryl;
wherein in formula (6), R₃ is a hydrogen atom or a hydroxyl-protecting group; and
wherein in formula (6′), R₂′ is a suitable amino-protecting group and R₃′ and R₃″, identical or different, are a hydrogen atom or a methyl group; or R₂′ and one of R₃′ and R₃″ form together a πr-bond and the other of R₃′ and R₃″ is a t-butoxy group or a phenyl group.

A fifth broad aspect of the present invention preferably provides compounds of formula (8) or (8′) which are defined as follows:

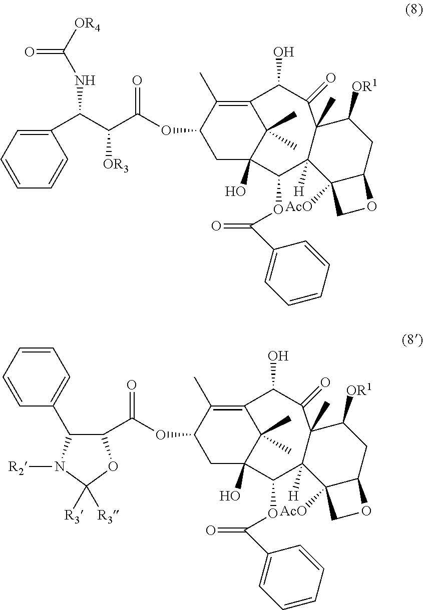

wherein R₁ is a hydrogen atom of a suitable hydroxyl-protecting group;
wherein in formula (8′) R₂′ is a suitable amino-protecting group and R₃′ and R₃″, identical or different, are a hydrogen atom or a methyl group; or R₂′ and one of R₃′ and R₃″ form together a π-bond and the other of R₃′ and R₃″ is a t-butoxy group or a phenyl group;
wherein in formula (8) R₃ is a hydrogen atom or a suitable hydroxyl-protecting group; and
wherein in formula (8) R₄ is a hydrogen atom, a linear C₁-C₂₀ alkyl, a branched C₃-C₂₀ alkyl group, a C₁-C₂₀ acyl group, a C₁-C₂₀ halogenated acyl group, a C₃-C₁₂ cycloalkyl, a C₁-C₁₂ heterocyclyl, a C₂-C₂₀ alkenyl, a C₂-C₂₀ alkynyl, a C₆-C₁₂ aryl, a C₆-C₂₀ aralkyl, a C₁-C₂₀ alkyloxy C₆-C₂₀ alkylaryl, a C₁-C₁₂ heteroaryl, a C₂-C₂₀ alkylheterocyclyl or a C₂-C₂₀ alkylheteroaryl,
said alkyl, cycloalkyl, heterocyclyl, alkenyl, alkynyl, aryl, aralkyl, alkylaryl, heteroaryl, alkylheterocyclyl and alkylheteroaryl being unsubstituted or substituted with at least one substituent, each of said substituent(s) being selected from the group consisting of F, Cl, Br, I, OH, SH, NH₂, NO₂, CN, CF₃, —SH, —OCH₂Ph, —OPh, —SCH₃, —SPh, —SCH₂Ph, —COOH, —COOR₆ (in which R₆ is a C₁-C₆ alkyl), linear C₁-C₂₀ alkyl, branched C₃-C₂₀ alkyl, C₆-C₁₂ aryl, C₂-C₂₀ alkenyl, C₁-C₂₀ alkoxy, C₂-C₂₀ alkynyl, C₆-C₂₀ aralkyl, C₆-C₁₂ aryl, C₃-C₈ cycloalkyl, C₁-C₂₀ aminoalkyl, C₆-C₁₂ aminoaryl, C₁-C₁₂ aminoheteroaryl, C₁-C₂₀ hydroxyalkyl, C₆-C₁₂ hydroxyaryl, C₁-C₁₂ hydroxyheteroaryl, C₁-C₁₂ heterocyclyl, C₁-C₁₂ heteroaryl, C₂-C₂₀ alkylheterocyclyl and C₂-C₂₀ alkylheteroaryl.

A sixth broad aspect of the present invention preferably provides a compound of formula (10) which is defined as follows:

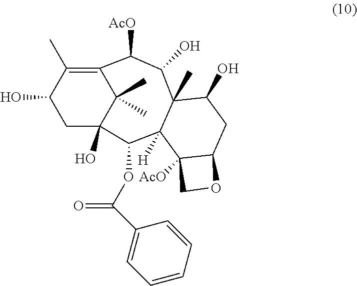

A seventh broad aspect of the present invention preferably provides compounds of (11) which are defined as follows:

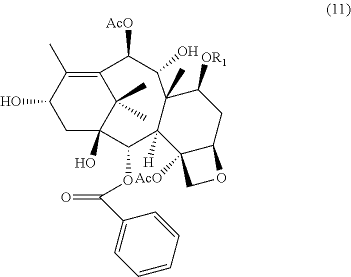

wherein R₁ is a hydrogen atom or a suitable hydroxyl-protecting group.

A eighth broad aspect of the present invention preferably provides compounds of formula (12) which are defined as follows:

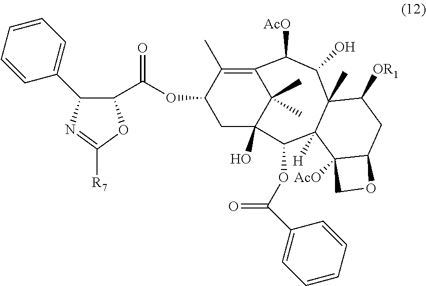

wherein R₁ is a hydrogen atom or a suitable hydroxyl-protecting group; and
wherein X is a Boc group or a benzyl group.

A ninth broad aspect of the present invention preferably provides compounds of formula (13) which are defined as follows:

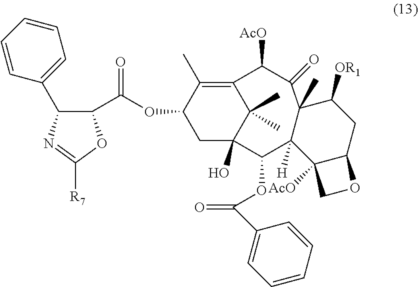

wherein R₁ is a hydrogen atom or a suitable hydroxyl-protecting group; and
wherein R₇ is C₂-C₁₀ alkynyl, C₁-C₁₀ alkyl, C₂-C₁₀ alkenyl, C₁-C₁₀ alkoxy, C₆-C₁₂ aryl or C₅-C₁₂ heteroaryl, preferably R₇ is a t-butoxy group or a phenyl group.

A tenth broad aspect of the present invention preferably provides compounds of formula (14) which are defined as follows:

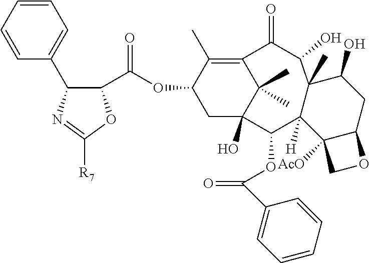

wherein R₇ is C₂-C₁₀ alkynyl, C₁-C₁₀ alkyl, C₂-C₁₀ alkenyl, C₁-C₁₀ alkoxy, C₆-C₁₂ aryl or C₅-C₁₂ heteroaryl, preferably a t-butoxy group or a phenyl group.

An eleventh broad aspect of the invention is to provide a process for the preparation of docetaxel and/or paclitaxel.

A twelfth broad aspect of the present invention preferably provides a process for preparing docetaxel and derivative thereof, comprising a step of intramolecular isomerization a compound of formula (4):

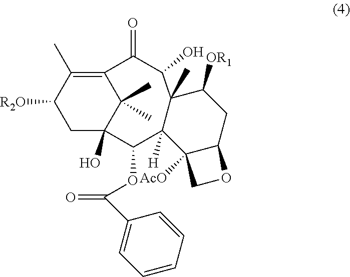

wherein R₁ is a hydrogen atom or a suitable hydroxyl-protecting group;
wherein R₃ is a hydrogen atom or a suitable protecting group for a hydroxyl group; and
wherein R₄ is a hydrogen atom, a C₁-C₂₀ alkyl linear or branched, C₁-C₂₀ acyl group, C₁-C₂₀ halogenated acyl group, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₁-C₂₀ alkyloxy C₆-C₂₀ alkylaryl, C₁-C₁₂ heteroaryl, C₂-C₂₀ alkylheterocyclyl or C₂-C₂₀ alkylheteroaryl, said alkyl, cycloalkyl, heterocyclyl, alkenyl, alkynyl, aryl, aralkyl, alkylaryl, heteroaryl, alkylheterocyclyl, and alkylheteroaryl are unsubstituted or substituted with at least one substituent, each of said substituent(s) being chosen from F, Cl, Br, I, OH, SH, NH₂, NO₂, CN, CF₃, —SH, —OCH₂Ph, —OPh, —SCH₃, —SPh, —SCH₂Ph, —COOH, —COOR₆ (in which R₆ is a C₁-C₆ alkyl), C₁-C₂₀ alkyl linear, C₁-C₂₀ alkyl branched, C₆-C₁₂ aryl, C₂-C₂₀ alkenyl, C₁-C₂₀ alkoxy, C₂-C₂₀ alkynyl, C₆-C₂₀ aralkyl, C₆-C₁₂ aryl, C₃-C₈ cycloalkyl, C₁-C₂₀ aminoalkyl, C₆-C₁₂ aminoaryl, C₁-C₁₂ aminoheteroaryl hydroxyalkyl, C₆-C₁₂ hydroxyaryl, C₁-C₁₂ hydroxyheteroaryl, C₁-C₁₂ heterocyclyl, C₁-C₁₂ heteroaryl, C₂-C₂₀ alkylheterocyclyl and C₂-C₂₀ alkylheteroaryl, by addition of at least one intramolecular isomerization agent to transform said compound of formula (4) into a compound of formula (7):

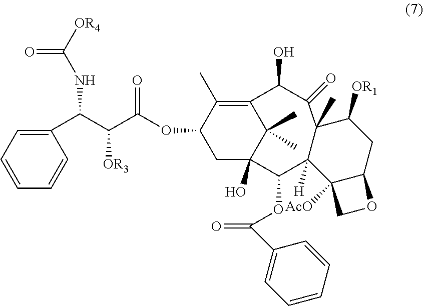

wherein R₁, R₃ and R₄ are as defined hereinabove, followed if necessary by a deprotection step removing eventual protective groups defined by R₁, R₃ and R₄. More preferably R₁ is a t-butyldiphenyl silyl, R₃ is ethoxyethyl, R₄ is t-butyloxyl, and the deprotection step is carried out in with HF.

A thirteenth broad aspect of the present invention preferably provides an improvement in a process for the preparation of aforesaid intermediates of formula (2), (3), (4), (5), (6), (6′), (8), (8′). (10). (11), (12), (13) and (14).

A fourteenth broad aspect of the present invention preferably provides, a process for the preparation of a compound of formula (4):

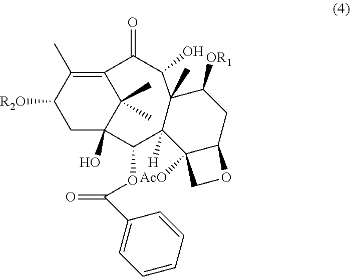

wherein R₁ is a hydrogen atom of a suitable hydroxyl-protecting group; and
wherein R₂ is a hydrogen atom or a suitable hydroxyl-protecting group;
said process comprising the step of oxidating a compound of formula (3):

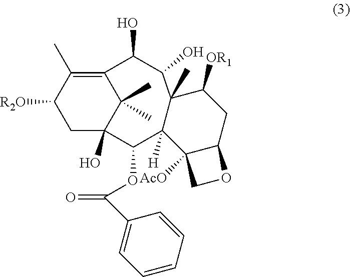

wherein R₁ and R₂ are as defined hereinbefore. More preferably, R₁ is t-butyldiphenyl silyl and R₂ is acetyl.

A fifteenth broad aspect of the present invention preferably provides a process for the preparation of a compound of formula (5):

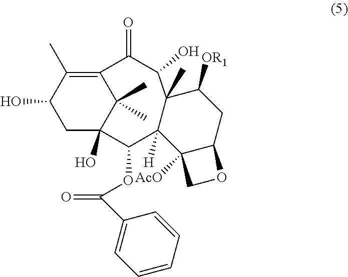

wherein R₁ is a hydrogen atom or a suitable hydroxyl-protecting group, said process comprising the step of removing the protecting group in the 13 position of a compound of formula (4):

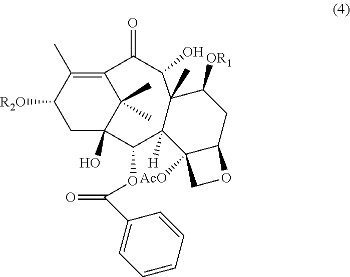

wherein R₁ is as defined hereinabove and R₂ is a suitable hydroxyl-protecting group. More preferably, R₂ is an acetyl and removing of the protecting group in 13 position is carried out with n-butyl lithium at −60° C.

A sixteenth broad aspect of the present invention preferably provides, a process for the preparation of a compound of formula (6):

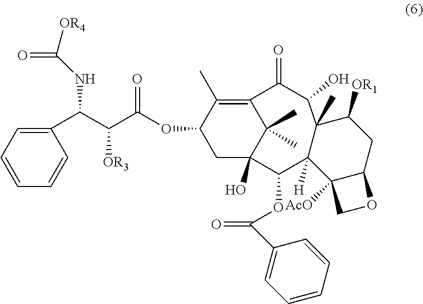

wherein R₁ is a hydrogen atom of a suitable hydroxyl-protecting group;
wherein R₃ is a hydrogen atom or a suitable hydroxyl-protecting group; and
wherein R₄ is a hydrogen atom, a C₁-C₂₀ alkyl linear or branched, C₁-C₂₀ acyl group, C₁-C₂₀ halogenated acyl group, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₁-C₂₀ alkyloxy C₆-C₂₀ alkylaryl, C₁-C₁₂ heteroaryl, C₂-C₂₀ alkylheterocyclyl, or C₂-C₂₀ alkylheteroaryl, said alkyl, cycloalkyl, heterocyclyl, alkenyl, alkynyl, aryl, aralkyl, alkylaryl, heteroaryl, alkylheterocyclyl and alkylheteroaryl are unsubstituted or substituted with at least one substituent, each of said substituent(s) being chosen from F, Cl, Br, I, OH, SH, NH₂, NO₂, CN, CF₃, —SH, —OCH₂Ph, —OPh, —SCH₃, —SPh, —SCH₂Ph, —COOH, —COOR₆ (in which R₆ is a C₁-C₆ alkyl), C₁-C₂₀ alkyl linear, C₁-C₂₀ alkyl branched, C₆-C₁₂ aryl, C₂-C₂₀ alkenyl, C₁-C₂₀ alkoxy, C₂-C₂₀ alkynyl, C₆-C₂₀ aralkyl, C₆-C₁₂ aryl, C₃-C₈ cycloalkyl, C₁-C₂₀ aminoalkyl, C₆-C₁₂ aminoaryl, C₁-C₁₂ aminoheteroaryl, C₁-C₂₀ hydroxyalkyl, C₆-C₁₂ hydroxyaryl, C₁-C₁₂ hydroxyheteroaryl, C₁-C₁₂ heterocyclyl, C₁-C₁₂ heteroaryl, C₂-C₂₀ alkylheterocyclyl and C₂-C₂₀ alkylheteroaryl;
said process comprising the step of reacting a precursor of side chain of formula:

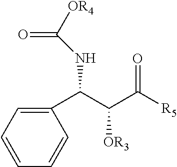

wherein R₃ and R₄ are as defined hereinabove, and R₅ is a radical suitable to add said side chain in the 13 position of the compound of formula (5):

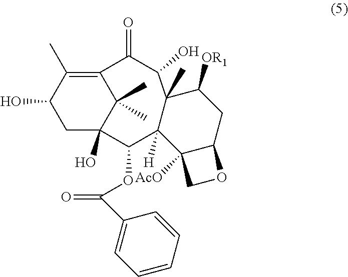

wherein R₁ is as defined hereinabove, to form said compound of formula (6). Preferably, R₁ is a t-butyldiphenyl silyl, R₃ is ethoxyethyl, R₄ is t-butyloxyl, and R₅ is a hydroxyl group.

A seventeenth broad aspect of the present invention preferably provides a process for the preparation of compounds of formula (6′):

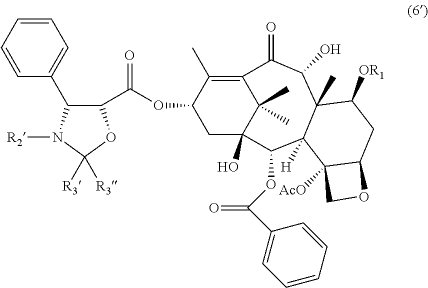

wherein R₁ is a hydrogen atom of a suitable hydroxyl-protecting group; and
wherein in formula (6′), R₂′ is a suitable amino-protecting group and R₃′ and R₃″, identical or different, are a hydrogen atom or a methyl group; or R₂′ and one of R₃′ and R₃″ form together a π-bond and the other of R₃′ and R₃″ is a t-butoxy group or a phenyl group, said process comprising the step of reacting a precursor of side chain of formula:

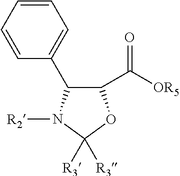

wherein R₂′, R₃′ and R₃″ are as defined hereinabove, and R₅ is a radical suitable to add said side chain in the 13 position of the compound of formula (5):

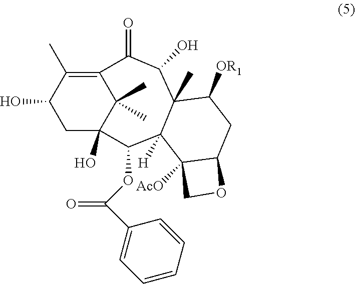

wherein R₁ is as defined hereinabove, to form said compound of formula (6′).

A eighteenth broad aspect of the present invention preferably provides a process for the preparation of compounds of formula (3):

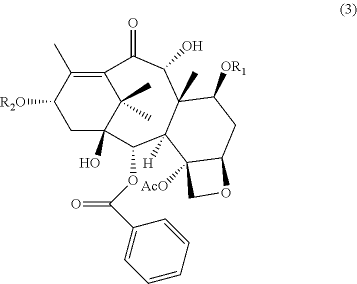

wherein R₁ is a hydrogen atom of a suitable hydroxyl-protecting group; and
wherein R₂ is a hydrogen atom or a suitable hydroxyl-protecting group,
said process comprising a step of reacting a compound of formula (2):

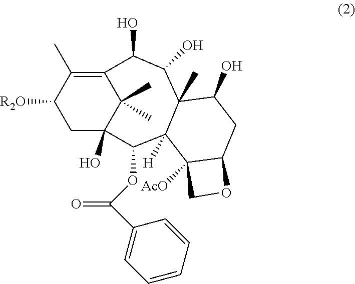

wherein R₂ is as defined hereinabove, in the presence of an agent suitable to protect the hydroxyl in the 7 position. Preferably, the agent suitable to protect the hydroxyl group in the 7 position is t-butyldiphenylsilyl chloride.

A nineteenth broad aspect of the present invention preferably provides a process for the preparation of compounds of formula (2):

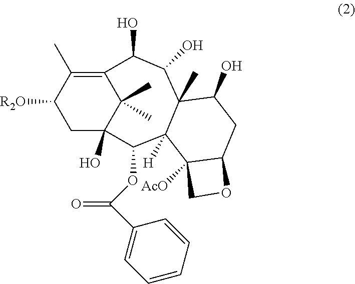

wherein R₂ is a hydrogen atom or a suitable hydroxyl-protecting group;
said process comprising the deacetylation of the acetyl group in position 10 of the 9-dihydro-13-acetylbaccatin III of formula (1):

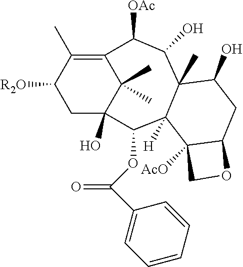

wherein R₂ is as defined hereinabove.

A twentieth broad aspect of the present invention preferably provides a process for the preparation of compounds of formula (8):

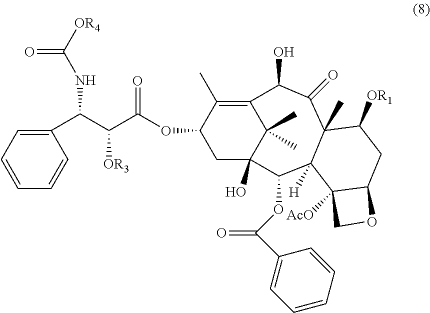

wherein R₁ is a hydrogen atom of a suitable hydroxyl-protecting group;
wherein R₃ is a hydrogen atom or a suitable hydroxyl-protecting group; and
wherein R₄ is a hydrogen atom, a C₁-C₂₀ alkyl linear, C₃-C₂₀ alkyl branched, C₁-C₂₀ acyl group, C₁-C₂₀ halogenated acyl group, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₁-C₂₀ alkyloxy C₆-C₂₀ alkylaryl, C₁-C₁₂ heteroaryl, C₂-C₂₀ alkylheterocyclyl, or C₂-C₂₀ alkylheteroaryl,
said alkyl, cycloalkyl, heterocyclyl, alkenyl, alkynyl, aryl, aralkyl, alkylaryl, heteroaryl, alkylheterocyclyl, and alkylheteroaryl are unsubstituted or substituted with at least one substituent, each of said substituent(s) being selected from the group consisting of F, Cl, Br, I, OH, SH, NH₂, NO₂, CN, CF₃, —SH, —OCH₂Ph, —OPh, —SCH₃, —SPh, —SCH₂Ph, —COOH, —COOR₆ (in which R₆ is a C₁-C₆ alkyl), C₁-C₂₀ alkyl linear, C₃-C₂₀ alkyl branched, C₆-C₁₂ aryl, C₂-C₂₀ alkenyl, C₁-C₂₀ alkoxy, C₂-C₂₀ alkynyl, C₆-C₂₀ aralkyl, C₆-C₁₂ aryl, C₃-C₈ cycloalkyl, C₁-C₂₀ aminoalkyl, C₆-C₁₂ aminoaryl, C₁-C₁₂ aminoheteroaryl, C₁-C₂₀ hydroxyalkyl, C₆-C₁₂ hydroxyaryl, C₁-C₁₂ hydroxyheteroaryl, C₁-C₁₂ heterocyclyl, C₁-C₁₂ heteroaryl, C₂-C₂₀ alkylheterocyclyl and C₂-C₂₀ alkylheteroaryl. This process advantageously comprises a step of intramolecular isomerization a compound of formula (6):

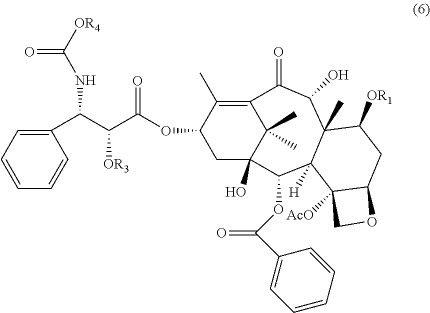

wherein R₁, R₃ and R₄ are as defined hereinabove. Preferably, R₁ is a t-butyldiphenyl silyl, R₃ is ethoxyethyl, R₄ is t-butyloxy. Preferably, the intramolecular isomerization is obtained by subjecting the compound of formula (6) to a guanidine base in methylene chloride.

A twentyfirst broad aspect of the present invention preferably provides a process for the preparation of a compound of formula (8′):

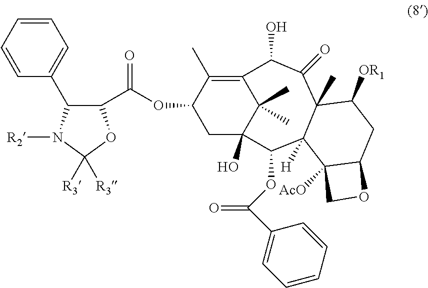

wherein R₁ is a hydrogen atom of a suitable hydroxyl-protecting group; and
wherein in formula (8′) R₂′ is a suitable amino-protecting group and R₃′ and R₃″, identical or different, are a hydrogen atom or a methyl group; or R₂′ and one of R₃′ and R₃″ form together a π-bond and the other of R₃′ and R₃″ is a t-butoxy group or a phenyl group. This process advantageously comprises a step of intramolecular isomerization a compound of formula (6′):

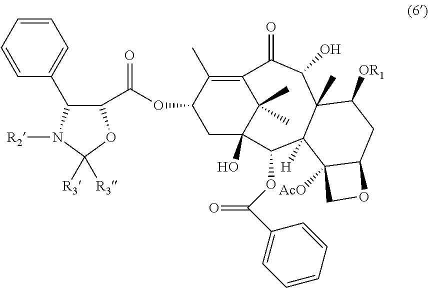

wherein R₁ is a hydrogen atom of a suitable hydroxyl-protecting group; and
wherein in formula (6′), R₂′ is a suitable amino-protecting group and R₃′ and R₃″, identical or different, are a hydrogen atom or a methyl group; or R₂′ and one of R₃′ and R₃″ form together a π-bond and the other of R₃′ and R₃″ is a t-butoxy group or a phenyl group.

A twentysecond broad aspect of the present invention preferably provides a process for the preparation of a compound of formula (10):

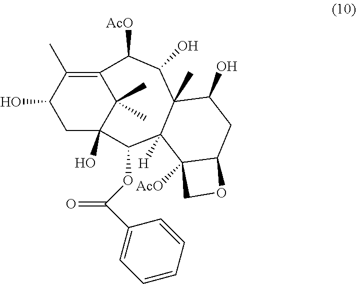

said process comprising the step of submitting a compound of formula:

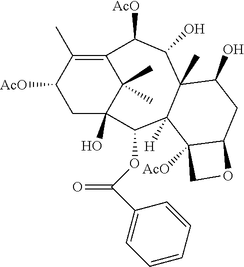

### to CH₃Li/n.BuLi in THF at −60° C.

A twentythird broad aspect of the present invention preferably provides a process for the preparation of a compound of formula (11):

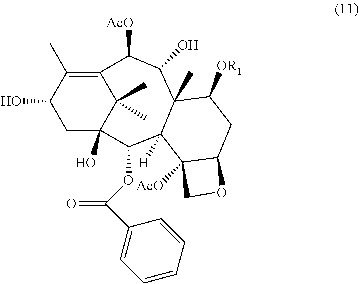

wherein R₁ is a hydrogen atom or a suitable hydroxyl-protecting group, said process comprising a step of protecting the hydroxyl group in position 7 in a compound of formula (10):

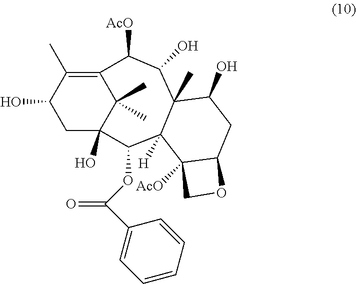

with a hydroxyl-protecting group.

A twentysecond broad aspect of the present invention preferably provides a process for the preparation of a compound of formula (12):

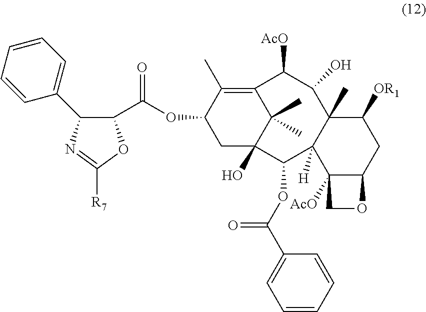

wherein R₁ is a hydrogen atom or a suitable hydroxyl-protecting group; and
wherein R₇ is C₂-C₁₀ alkynyl, C₁-C₁₀ alkyl, C₂-C₁₀ alkenyl, C₁-C₁₀ alkoxy, C₆-C₁₂ aryl or C₅-C₁₂ heteroaryl, preferably R₇ is a t-butoxy group or a phenyl group, said process comprising the step of reacting a compound of formula (11):

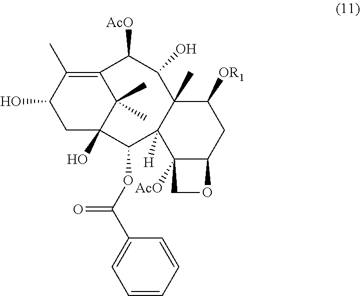

wherein R₁ is a hydrogen atom or a suitable protecting group for a hydroxyl group, with a compound of formula:

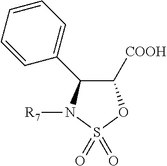

wherein X represents a radical of formula R₇—CO— where R₇ is C₂-C₁₀ alkynyl, C₁-C₁₀ alkyl, C₂-C₁₀ alkenyl, C₁-C₁₀ alkoxy, C₆-C₁₂ aryl or C₅-C₁₂ heteroaryl, preferably a t-butoxy group or a phenyl group, in the presence of DCG, DMAP and toluene at 70° C.

A twentyfifth broad aspect of the present invention preferably provides a process for the preparation of a compound of formula (13):

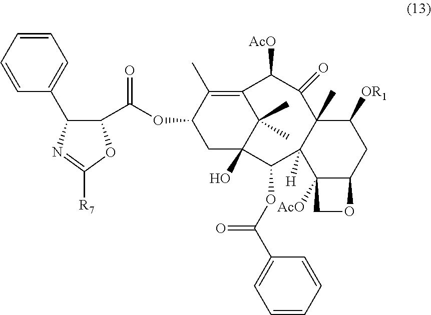

wherein R₁ is a hydrogen atom or a suitable hydroxyl-protecting group; and
wherein R₇ is C₂-C₁₀ alkynyl, C₁-C₁₀ alkyl, C₂-C₁₀ alkenyl, C₁-C₁₀ alkoxy, C₆-C₁₂ aryl or C₅-C₁₂ heteroaryl, preferably a t-butoxy group or a phenyl group, said process comprising a step submitting a compound of formula (12):

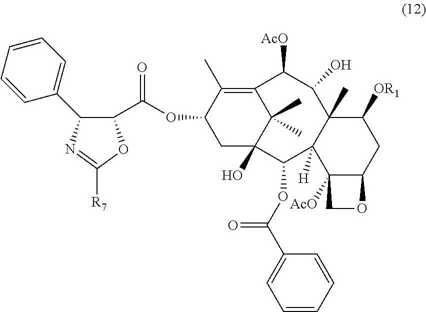

wherein R₁ and R₇ are as defined hereinabove, to the presence of TPAP and NMO.

The suitable hydroxyl-protecting groups can be any protecting group that would be used by a person skilled in the art to protect a hydroxyl group.

Such hydroxyl-protecting groups can be those described in Theodora W. Greene et al., Protective Groups in Organic Synthesis, Third Edition, John Wiley & Sons, Inc., 1999, pp. 17-21, which is hereby incorporated by reference.

The hydroxyl-protecting groups, for example, ethers (such as methyl), or substituted methyl ethers (such as methoxymethyl; methylthiomethyl; (phenyldimethylsilyl)methoxymethyl; benzyloxymethyl; ρ-methoxybenzyloxymethyl; p-nitrobenzyloxymethyl; o-nitrobenzyloxymethyl; (4-methoxyphenoxy)methyl; guaiacolmethyl; t-butoxymethyl; 4-pentenyloxymethyl; siloxymethyl; 2-methoxyethoxymethyl; 2,2,2-trichloroethoxymethyl; bis(2-chloroethoxy)methyl; 2-(trimethylsilyl)ethoxymethyl; menthoxymethyl; tetrahydropyranyl; 3-bromotetrahydropyranyl; tetrahydrothiopyranyl; 1-methoxycyclohexyl; 4-methoxytetrahydropyranyl; 4-methoxytetrahydrothiopyranyl; 4-methoxytetrahydrothiopyranyl s,s-dioxide; 1-[(2-chloro-4-methyl)phenyl]-4-methoxypiperidin-4-yl; 1-(2-fluorophenyl)-4-methoxypiperidin-4-yl; 1,4-dioxan-2-yl; tetrahydrofuranyl; tetrahydrothiofuranyl; 2,3,3a,4,5,6,7,7a-octahydro-7,8,8-trimethyl-4,7-methanobenzofuran-2-yl).

The hydroxyl-protecting groups, for example, substituted ethyl ethers (such as 1-ethoxyethyl; 1-(2-chloroethoxy)ethyl; 1-[2-(trimethylsilyl)ethoxy]ethyl; 1-methyl-1-methoxyethyl; 1-methyl-1-benzyloxyethyl; 1-methyl-1-benzyloxy-2-fluoroethyl; 1-methyl-1-phenoxyethyl; 2,2,2-trichloroethyl; 1,1-dianisyl-2,2,2-trichloroethyl; 1,1,1,3,3,3-hexafluoro-2-phenylisopropyl; 2-trimethylsilylethyl; 2-(benzylthio)ethyl; 2-(phenylselenyl)ethyl; t-butyl; allyl; propargy; ρ-chlorophenyl; ρ-methoxyphenyl; ρ-nitrophenyl; 2,4-dinitrophenyl; 2,3,5,6-tetrafluoro-4-(trifluoromethyl)phenyl; benzyl), substituted benzyl ethers (such as ρ-methoxybenzyl; 3,4-dimethoxybenzyl; o-nitrobenzyl; ρ-nitrobenzyl; ρ-halobenzyl; 2,6-dichlorobenzyl; ρ-cyanobenzyl; ρ-phenylbenzyl; 2,6-difluorobenzyl; ρ-acylaminobenzyl; ρ-azidobenzyl; 4-azido-3-chlorobenzyl; 2-trifluoromethylbenzyl; ρ-(methylsulfinyl)benzyl; 2- and 4-picolyl; 3-methyl-2-picolyl n-oxido; 2-quinolinylmethyl; 1-pyrenylmethyl; diphenylmethyl; p,p′-dinitrobenzhydryl; 5-dibenzosuberyl; triphenylmethyl; α-naphthyldiphenylmethyl; ρ-methoxyphenyldiphenylmethyl; di(ρ-methoxyphenyl)phenylmethyl; tri(ρ-methoxyphenyl)methyl; 4-(4′-bromophenacyloxy)phenyldiphenylmethyl; 4,4′,4″-tris(4,5-dichlorophthalimidophenyl)methyl; 4,4′,4″-tris(levulinoyloxyphenyl)methyl; 4,4′,4″-tris(benzoyloxyphenyl)methyl; 4,4′-dimethoxy-3″-[n-(imidazolylmethyl)]trityl; 4,4′-dimethoxy-3″-[n-(imidazolylethyl)carbamoyl]trityl; 1,1-bis(4-methoxyphenyl)-1′-pyrenyl methyl; 4-(17-tetrabenzo[a,c,g,i]fluorenylmethyl)-4,4″-dimethoxytrityl; 9-anthryl; 9-(9-phenyl)xanthenyl; 9-(9-phenyl-10-oxo)anthryl; 1,3-benzodithiolan-2-yl; benzisothiazolyl s,s-dioxido) silyl ethers (such as trimethylsilyl; triethylsilyl; triisopropylsilyl; dimethylisopropylsilyl; diethylisopropylsilyl; dimethylthexylsilyl; t-butyldimethylsilyl; t-butyldiphenylsilyl; tribenzylsilyl; tri-ρ-xylylsilyl; triphenylsilyl; diphenylmethylsilyl; phenyldimethylsilyl, di-t-butylmethylsilyl; tris(trimethylsilyl)silyl: sisyl; (2-hydroxystyryl)dimethylsilyl; (2-hydroxystyryl)diisopropylsilyl; t-butylmethoxyphenylsilyl; t-butoxydiphenylsilyl), esters (such as formate; benzoylformate; acetate; chloroacetate; dichloroacetate; trichloroacetate; trifluoroacetate; methoxyacetate; triphenylmethoxyacetate; phenoxyacetate; ρ-chlorophenoxyacetate; phenylacetate; ρ-p-phenylacetate; diphenylacetate; nicotinate; 3-phenylpropionate; 4-pentenoate; 4-oxopentanoate (levulinate); 4,4-(ethylenedithio)pentanoate; 5-[3-bis(4-methoxyphenyl)hydroxymethylphenoxy]levulinate; pivaloate; 1-adamantoate; crotonate; 4-methoxycrotonate; benzoate; ρ-phenylbenzoate; 2,4,6-trimethylbenzoate (mesitoate), carbonates (such as methylcarbonyl; methoxymethylcarbonyl; 9-fluorenylmethylcarbonyl; ethylcarbonyl; 2,2,2-trichloroethylcarbonyl; 1,1-dimethyl-2,2,2-trichloroethylcarbonyl; 2-(trimethylsilyl)ethylcarbonyl; 2-(phenylsulfonyl)ethylcarbonyl; 2-(triphenylphosphonio)ethylcarbonyl; isobutylcarbonyl; vinylcarbonyl; allylcarbonyl; ρ-nitrophenylcarbonyl; benzylcarbonyl; ρ-methoxybenzylcarbonyl; 3,4-dimethoxybenzylcarbonyl; o-nitrobenzylcarbonyl; ρ-nitrobenzylcarbonyl), carbonates cleaved by b-elimination (such as 2-dansylethyl; 2-(4-nitrophenyl)ethyl; 2-(2,4-dinitrophenyl)ethyl; 2-cyano-1-phenylethyl; s-benzyl thiocarbonate; 4-ethoxy-1-naphthyl; methyl dithiocarbonate), miscellaneous esters (such as 2,6-dichloro-4-methylphenoxyacetate; 2,6-dichloro-4-(1,1,3,3-tetramethylbutyl)phenoxyacetate; 2,4-bis(1,1-dimethylpropyl)phenoxyacetate; chlorodiphenylacetate; isobutyrate; monosuccinoate; (e)-2-methyl-2-butenoate (tigloate); o-(methoxycarbonyl)benzoate; ρ-p-benzoate; α-naphthoate; nitrate; alkyl n,n,n′,n′-tetramethylphosphorodiamidate; 2-chlorobenzoate; 4-bromobenzoate; 4-nitrobenzoate; 3′5′-dimethoxybenzoin; n-phenylcarbamate; borate; dimethylphosphinothioyl; 2,4-dinitrophenylsulfenate), and sulfonate (such as sulfate; allylsulfonate; methanesulfonate (mesylate); benzylsulfonate; tosylate; 2-[(4-nitrophenyl)ethyl]sulfonate).

According to particularly preferred aspects of the invention, R1, R2, R2′, R3, R3′, R3″ and R4 may have the following definitions:
R₁ may be a hydroxyl-protecting group of formula:

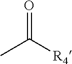

wherein R₄′ forms with the carbonyl a C₁-C₂₀ acyl group or a C₁-C₂₀ halogenated acyl group;
R₁ may be a t-butyldiphenyl silyl, diphenylmethylsilyl or phenyldimethylsilyl;
R₁ may be a phenyldimethylsilyl, R₂′ and R₃′ may form together a π-bond and R₃″ may be a t-butoxy;
R₁ may be a phenyldimethylsilyl, R₂′ may be a Boc, R₃′ and R₃″ may be a each methyl;
R₁ may be a phenyldimethylsilyl, R₂′ may be a benzyl, R₃′ and R₃″ may be a each methyl;
R₁ may be a phenyldimethylsilyl, R₂′ and R₃′ may form together a π-bond and R₃″ may be phenyl;
R₁ may be a phenyldimethylsilyl, R₂ may be absent, R₃ may be a hydrogen atom and R₃′ may be absent;
R₁ may be a phenyldimethylsilyl, R₂ may be absent, R₃ may be a ethoxyethyl and R₃′ may be absent;
R₁ may be a hydrogen atom and R₂ may be a acetyl;
R₂ may be a acetyl;
R₃ may be a ethoxyethyl;
R₄ may be a C₁-C₆ alkyl, phenyl, t-butyloxyl, a C₂-C₆ alkenyl, tetrahydrofuranyl or tetrahydropyranyl;
R₄ may be a t-butyloxyl; or
R₁ may be a t-butyldiphenyl silyl, diphenylmethylsilyl or phenyldimethylsilyl, R₃ may be a ethoxyethyl, and R₄ may be a t-butyloxyl.

### OTHER FEATURES OF THE INVENTION

The foregoing summarizes the principal features of the invention and some of its optional aspects. The invention may be further understood by the description of the preferred embodiments which now follow.

### DESCRIPTION OF DETAILED INVENTION

The following are non-limiting examples of the process of aspects of the present invention.

### Example 1

### Process for converting 9-Dihydro-13-acetylbaccatin III to Docetaxel

### Step 1: Remove 10-Acetyl Group

50 Grams of 9-dihydro-13-acetylbaccatin III was dissolved in 1 litre of acetonitrile, after stirred for 5 minutes then one mole equivalent of sodium methoxide was added, the mixture was stirred at room temperature for 5 hours or until the reaction was completed (monitored by TLC). After normal work up the organic phase was collected and concentrated under vacuum, the white powder like product was identified by H-NMR as 10-deacetyl-9-dihydro-13-acetylbaccatin III (yield: 96%).

### Reaction Scheme of Step 1

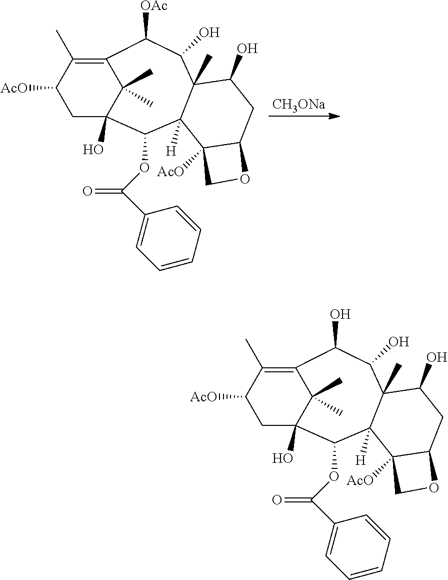

### Step 2: Protection of 7-Hydroxyl Group

The material from step 1 was dissolved in dichloromethane, imidazole and n-tetrabutylammonium iodine were added, the mixture was stirred at 0° C. for 10 minutes, then 3 mole equivalent of t-butyldiphenylsilyl chloride was added dropwise. The mixture was stirred for 1 hour then the temperature was warmed to about 30° C. and kept overnight at this temperature. The process was monitored by TLC, after work up the product was obtained as white powder and identified as 7-TBDPS-9-dihydro-10-deacetyl-13-acetylbaccatin III by H-NMR. Yield: 90%

### Reaction Scheme of Step 2

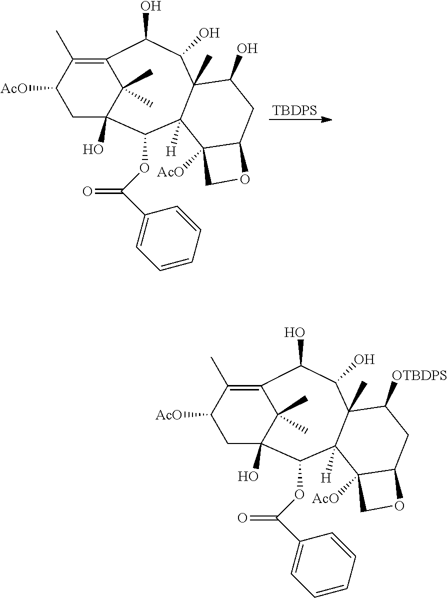

### Step 3: Oxidation

The product from step 2 was dissolved in acetonitrile and stirred at room temperature (30° C.) until the solid completely dissolved. 1.5 Mole equivalent NMO and 0.05% (mole equivalent) of TPAP, and some 4A molecular shiver were added. The mixture was stirred at 40° C. for 4 hours and monitored by TLC. After the reaction was completed then it was stopped by adding water. The product was extracted with dichloromethane. The dichloromethane phase was then concentrated to dryness under vacuum, the product was obtained as white solid, and identified as 9-dihydro-10-ketone-13-acetylbaccatin III by H, and C-NMR as well as 2D HMQC.

### Reaction Scheme of Step 3

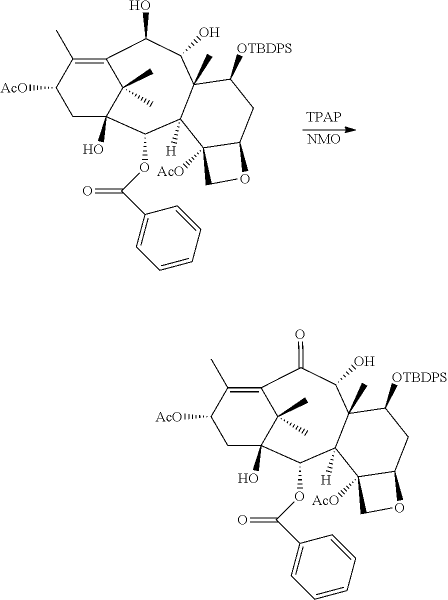

### Step 4: Deacetylation at 13-Position

To a solution of the product obtained from step 3 in tetrahydrofuran (THF) stirred at −60° C. under nitrogen was added n-BuLi (1M in hexane) dropwise. After 20 minutes the reaction was shown to be completed by TLC analysis. The reaction was quenched by adding of brine and dichloromethane. The organic phase was collected and evaporated to dryness under vacuum. The solid was dissolved in small amount methylene chloride and purified by flash column chromatography using hexane:ethyl acetate (2:1). The product 7-TBDPS-9-dihydro-10-ketonebaccatin III was obtained as white solid and identified by H-NMR.

### Reaction Scheme of Step 4

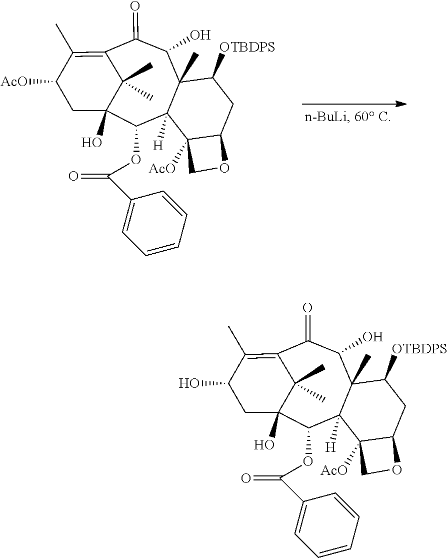

### Step 5: Attaching Docetaxel Side Chain

The product obtained from step 4 was dissolved in THF and stirred at −60° C. under nitrogen, lithium hexamethyldisilazide (LiHMDS, 1M in THF) was added dropwise. The mixture was stirred for 10 minutes then 1.5 equivalent of docetalxel side chain precursor was added, and then kept stirred at −60° C. for 1 hour. Then the mixture was warmed to 0° C. until the reaction was completed. Work up as normal, the product was obtained as white solid.

### Reaction Scheme of Step 5

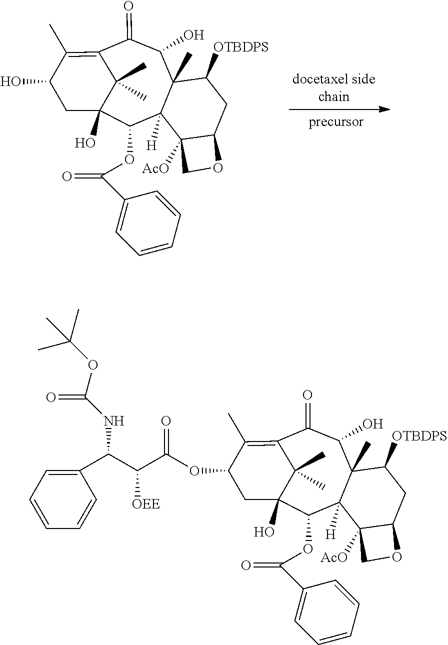

### Step 6: Intramolecular Isomerization

To a solution of the material obtained from Step 5 in methylene chloride stirred at room temperature was added 2 mole equivalent of guanidine base. The reaction mixture was stirred at room temperature for 20 minutes, one additional batch of guanidine base was added. After TLC shown that the reaction was completed, the mixture was concentrated and was directly purified through flash chromatography. The fractions which contained the desired product were combined and concentrated to dryness. The dried material was dissolved in toluene and 3 equivalent of 1,8-diazabicyclo[5,4,0]undec-7-ene (DBU) was added. The mixture was stirred at 80° C. for about 2 hours and then quenched with a saturated solution of NaHCO3 and brine. The mixture was extracted with EtOAc and washed with dilute HCl. The organic layer was then dried with anhydride Na₂SO₄ and concentrated to dryness then purified by flash chromatography.

### Reaction Scheme of Step 6

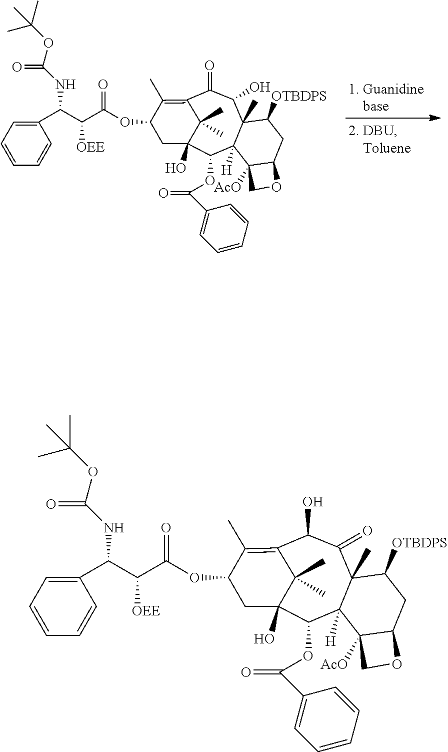

### Step 7: Deprotection

The product from step 6 was dissolved 1% HF in ethanol and stirred at room temperature for 4 hours and monitored by TLC. After TLC shown that the reaction was completed, the mixture was quenched by the adding of pH 7 phosphate buffer and partitioning between water and methylene chloride. The organic layer was separated, dried, and evaporated. The residue was purified by flash chromatography to give docetaxel as a white powder which identified by H-NMR and HPLC through an authentic sample.

### Reaction Scheme of Step 7

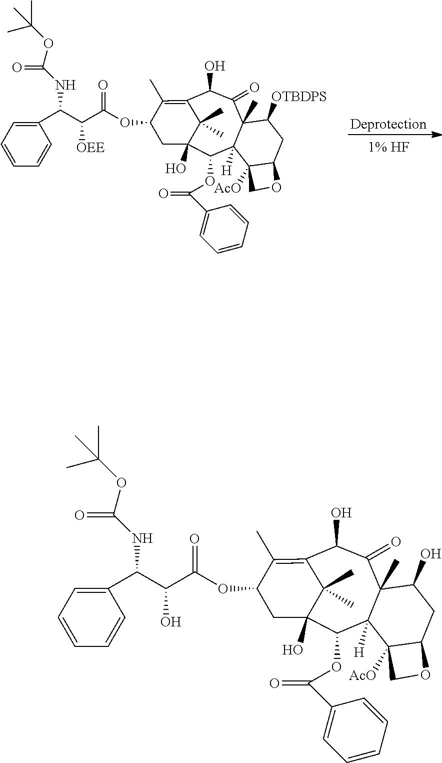

### Example 2

### Process for converting 9-Dihydro-13-acetylbaccatin III to Docetaxel

### Step 1, Remove 10-Acetyl Group

100 Grams of starting material, 9-dihydro-13-acetylbaccatin III, were dissolved in 1 liter of THF, after stirred for 5 minutes then 10 g of sodium methoxide was added, the mixture was stirred at room temperature for 5 hours or until the reaction was completed (monitored by TLC). After normal work up the organic phase was collected and concentrated under vacuum, the residue was crystallized from methanol to yield 86 grams of white solid. The white powder like product was identified by ¹H-NMR as 10-deacetyl-9-dihydro-13-acetylbaccatin III (yield: approx. 95%).

### Reaction Scheme of Step 1

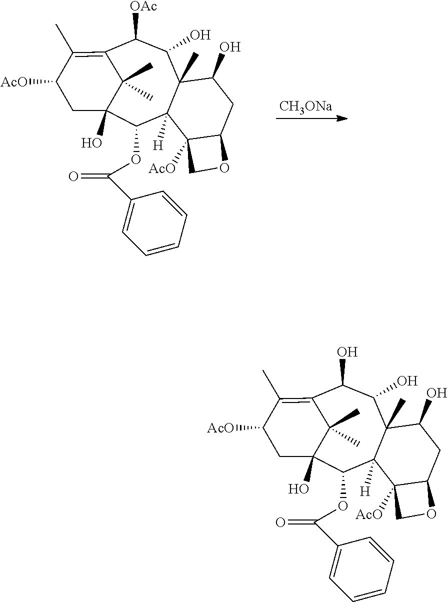

### Step 2, Protection of 7-Hydroxyl Group

The material from step 1 was dissolved in 700 ml of DMF, 2 mole of equivalent imidazole was added, the mixture was stirred at 0° C. for 10 minutes, then 2.5 mole equivalent of dimethylphenylsilyl chloride was added dropwise. The mixture was stirred for 1 hour then the temperature was warmed to about 20° C. and kept overnight at this temperature. The process was monitored by TLC, after work up the crude products were crystallized from acetone/hexanes mixed solvents. 91 Grams of crystal like product was obtained as white powder and identified as 7-DMPS-9-dihydro-10-deacetyl-13-acetylbaccatin III by ¹H-NMR. Yield: (approx. 90%), the only by-product was identified as 7,10-di-DMPS-9-dihydro-10-deacetylbaccatin III (approx. 10%). The final material can be used for next step reaction without pre-separation and purification through flash column chromatography.

### Reaction Scheme of Step 2

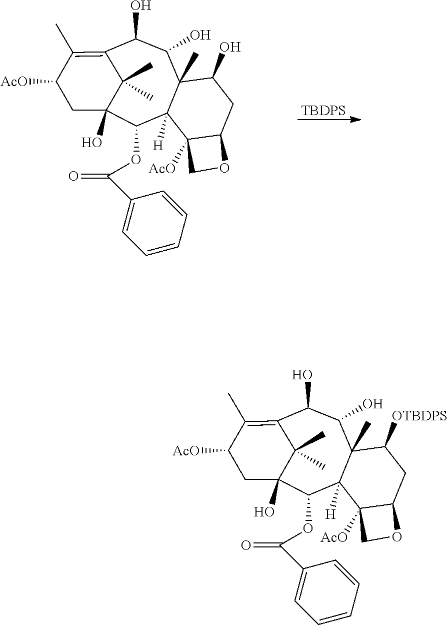

### Step 3: Oxidation

, The product from step 2 was dissolved in 1 liter of acetonitrile and stirred at room temperature (25° C.) until the solid completely dissolved. 4 Mole equivalent NMO and 0.05% (mole equivalent) of TPAP, and some 4A molecular shiver were added. The mixture was stirred at room temperature overnight. After the reaction was completed which was stopped by adding water. The product was extracted with dichloromethane. The dichloromethane phase was then concentrated to dryness under vacuum, the product was purified through flash column chromatography. The material obtained as white solid (85 g, yield: 93%), and identified as 7-DMPS-9-dihydro-10-keto-13-acetylbaccatin III by ¹H, and ¹³C-NMR as well as 2D HMQC.

### Reaction Scheme of Step 3

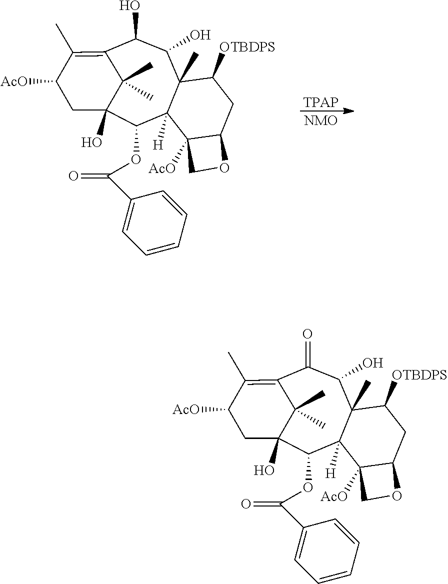

### Step 4: Deacetylation at 13-Position

The product obtained from step 3 was dissolved into 700 ml of 15% N₂H₄ (hydrazine monohydrate) in MeOH, the mixture was stirred at room temperature under nitrogen for 5 hours. After the reaction was shown to be completed by TLC analysis, the reaction was quenched by adding of brine and ethyl acetate. The organic phase was collected and evaporated to dryness under vacuum. The product 7-DMPS-9-dihydro-10-ketobaccatin III was obtained as white solid and identified by ¹H-NMR (73 g, yield: approx. 86%).

### Reaction Scheme of Step 4

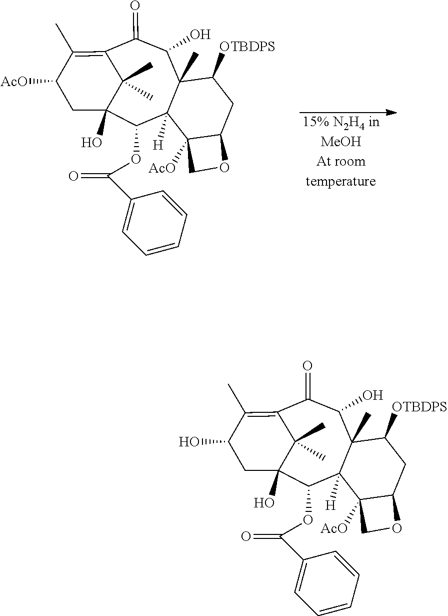

### Step 5: Attaching Docetaxel Side Chain

The product obtained from step 4 was dissolved in 500 ml of THF and stirred at −65° C. under nitrogen, 3 equivalent of docetaxel side chain precursors were then added. The mixture was stirred for 10 minutes before lithium hexamethyldisilazide (LiHMDS, 1M in THF) was added dropwise. Then the mixture was kept stirred at −60° C. for 1 hour then warmed to 0° C. until the reaction was completed. Work up as normal, the protected docetaxel derivative was obtained as white solid.

### Reaction Scheme of Step 5

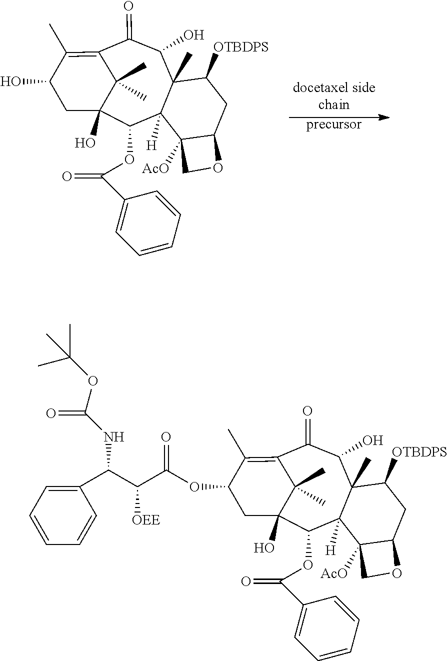

### Step 6: Intramolecular Isomerization

To a solution of the material obtained from Step 5 in THF stirred at −70° C. was added 1.5 mole equivalent of t-BuOK. The reaction mixture was stirred at this low temperature for 15 minutes. After TLC shown that the reaction was completed, the mixture was quenched by adding of brine and ethyl acetate. The organic phase was washed with dilute HCl, collected and concentrated, then purified through flash chromatography. The intermediate was obtained as white solid.

### Reaction Scheme of Step 6

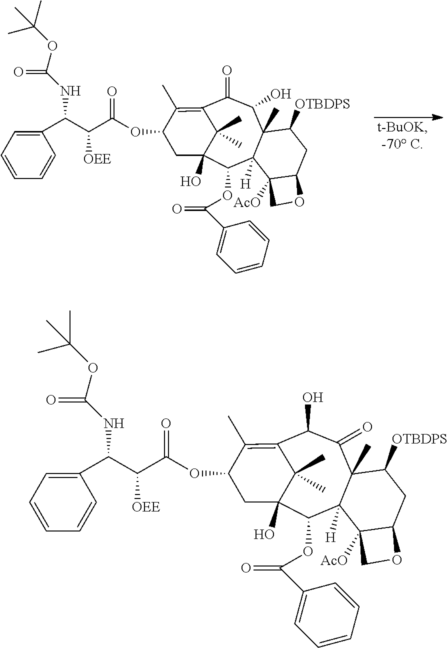

### Step 7: Deprotection

The products from step 6 was dissolved 10% HF in ethanol, some pyridine was added and stirred at room temperature for 4 hours and monitored by TLC. After TLC shown that the reaction was completed, the mixture was quenched by the adding of 5% NaHCO₃ solution and partitioning between water and ethyl acetate. The organic layer was separated, dried, and evaporated. The residue was purified by flash chromatography to give docetaxel as white powder which identified by ¹H-NMR and HPLC through comparison with an authentic sample.

### Reaction Scheme of Step 7

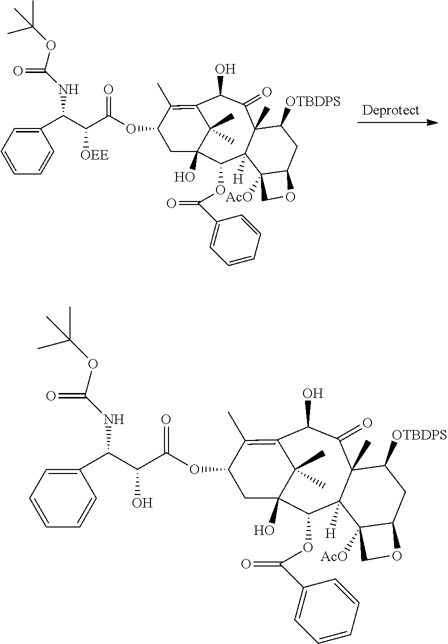

### Example 3

### Process for converting 9-Dihydro-13-acetylbaccatin III to Paclitaxel

The starting material 7-DMPS-9-dihydro-10-ketobaccatin III (Compound 5) obtained from Example 2 step 4 was used for this experiment.

### 1. Attaching Paclitaxel Side Chain

The product 7-DMPS-9-dihydro-10-ketobaccatin III was dissolved in THF and stirred at −65° C. under nitrogen, then 6 equivalent of paclitaxel side chain precursors were added. The mixture was stirred for 10 minutes before lithium hexamethyldisilazide (LiHMDS, 1M in THF) was added dropwise. Then the mixture was kept stirred at −60° C. for 1 hour then warmed to 0° C. until the reaction was completed. Work up as normal, the product was obtained as white solid.

### 2. Intramolecular Isomerization

To a solution of the material obtained from 1 in THF stirred at −70° C. was added 1.5 mole equivalent of t-BuOK. The reaction mixture was stirred at this low temperature for 15 minutes. After TLC shown that the reaction was completed, the mixture was quenched by adding of brine and ethyl acetate. The organic phase was washed with dilute HCl, collected and concentrated, then purified through flash chromatography. The product was obtained as white solid.

### 3. Attaching 10-Acetyl Group for Paclitaxel

The material from step 2 was dissolved in THF, and Ac₂O and CeCl₃ were added, the mixture was stirred at room temperature until the reaction was completed, then workup as normal, the product was obtained as a white powder.

### 4. Deprotection

The products from step 3 was dissolved 10% HF in ethanol, some pyridine was added and stirred at room temperature for 4 hours and monitored by TLC. After TLC shown that the reaction was completed, the mixture was quenched by the adding of 5% sodium bicarbonate solution and then partitioned between water and ethyl acetate. The organic layer was separated, dried, and evaporated. The residue was purified by flash chromatography to give paclitaxel as white powder which identified by ¹H-NMR and HPLC through comparison with an authentic sample.

### Example 4

### Process for converting 9-Dihydro-13-acetylbaccatin III to Docetaxel

The starting material 7-DMPS-9-dihydro-10-ketobaccatin III (Compound 5) can be obtained according the chemical reaction mentioned above in Example 2, step 4.

### 1. Attaching a Docetaxel Side Chain Precursor

The product 7-DMPS-9-dihydro-10-ketobaccatin III (20 g) and 12 g of oxazolidine acid of the formula (11) were dissolved in toluene, then 2.5 mole equivalent of dicyclohexylcarbodiimide (DCC) and 1 mole equivalent of DMAP was added, the mixture were stirred at 70° C. under nitrogen until the reaction was completed. At the end, the reaction mixture was filtered and the filtrate was diluted with ethyl acetate and water. The organic layer was washed with water, 5% sodium bicarbonate solution, and then dried over anhydrate sodium sulfate. Concentration of the organic phase under vacuum afforded 25 g of off white solid. The crude product was purified through flash chromatography and obtained as white solid.

### 2. Intramolecular Isomerization (in Acidic Condition)

The products from step 1 were dissolved into 200 ml of 40% HF in acetonitrile, then 10% pyridine was added. The mixture was stirred at room temperature, the reaction was monitored by TLC. After TLC shown that the reaction was completed, the mixture was quenched by the adding of 5% NaHCO₃ buffer and partitioned between water and ethyl acetate. The organic layer was separated, dried, and evaporated. The residue was purified by flash chromatography to give 13-(3′-N-Boc-4′-phenyl-2′,2′-dimethyl-oxazolidine-5′-carbonyloxy)-10-deacetylbaccatin III as white powder.

### 3. Deprotection

The product from step 2 was dissolved 200 ml of 80% HCOOH in acetonitrile and stirred at room temperature for 5 hours, the progress of the reaction was monitored by TLC. After TLC shown that the reaction was completed, the mixture was quenched by the adding saturated NaHCO₃ then partitioned between water and ethyl acetate. The organic layer was separated, dried with anhydrous Na₂SO₄, and evaporated. The residue was purified by flash chromatography to give the amino alcohol intermediate.

To the amino alcohol intermediate dissolved in 200 ml of THF was added 1.5 equivalent of di-tert-butyldicarbonate followed by 1 equivalent of DMAP. The mixture was stirred at room temperature until the starting material was disappeared as monitored by TLC. The reaction was quenched by adding of 5% NaHCO₃ and partitioning between water and ethyl acetate. The combined organic phase was washed with brine and dried by anhydrous Na₂SO₄, then concentrated under vacuum to afford crude docetaxel which was purified through flash column chromatography to obtained docetaxel as white powder which identified by ¹H-NMR and HPLC through comparison with an authentic sample.

### Example 5

### Process for converting 9-Dihydro-13-acetylbaccatin III to Paclitaxel

### 1. Attaching Paclitaxel Side Chain

Paclitaxel side chain precursor can be attached to 7-DMPS-9-dihydro-10-ketobaccatin III by using of 7-DMPS-9-dihydro-10-ketobaccatin III and oxazolidine acid of the formula (11) according the method disclosed above in Example 4.

13-(3′-N-Bz-4′-phenyl-2′,2′-dimethyl-oxazolidine-5′-carbonyloxy)-10-deacetylbaccatin III can be obtained by the same intramolecular isomerization method disclosed above in Example 4 step 2.

### 2. Attaching 10-Acetyl Group for Paclitaxel

13-(3′-N-Bz-4′-phenyl-2′,2′-dimethyl-oxazolidine-5′-carbonyloxy)-10-deacetylbaccatin III was dissolved in THF, and Ac₂O and CeCl₃ were added, the mixture was stirred at room temperature over night or until the reaction was completed, then workup as normal, the product was obtained as a white powder.

### 3. Deprotection

The product from step 2 was dissolved 200 ml of 80% HCOOH in acetonitrile and stirred at room temperature for 5 hours, the progress of the reaction was monitored by TLC. After TLC shown that the reaction was completed, the mixture was quenched by the adding saturated NaHCO₃ then partitioned between water and ethyl acetate. The organic layer was separated, dried with anhydrous Na₂SO₄, and evaporated. The residue was purified by flash chromatography to give paclitaxel as a white powder, which identified by ¹H-NMR and HPLC through comparison with an authentic sample.

### Example 6

### Process for converting 9-Dihydro-13-acetylbaccatin III to Docetaxel

The starting material 7-DMPS-9-dihydro-10-ketobaccatin III (Compound 5) can be obtained according the chemical reaction mentioned above in Example 2, step 4.

### 1. Attaching a Docetaxel Side Chain Precursor

The product 7-DMPS-9-dihydro-10-ketobaccatin III (10 g) and 6 g of 3-N-Boc-4-phenyl-(4S,5R)-2,2-dioxo-1,2,3-oxathiazolidine carboxylic acid of the formula (11′) were suspended in anhydrous toluene, then 4 mole equivalent of dicyclohexylcarbodiimide (DCC) and 1 mole equivalent of DMAP was added, the mixture were stirred at 70° C. under argon until TLC showed the formation of a new product. At the end, the reaction mixture was filtered and the filtrate was diluted with ethyl acetate. The organic layer was washed with water, 5% sodium bicarbonate solution, and then dried over anhydrate sodium sulfate. Concentration of the organic phase under vacuum afforded 10 g of off white solid. The crude product was purified through flash chromatography and obtained as white solid.

### 2. Intramolecular Isomerization (in Acidic Condition)

The products from step 1 were dissolved 100 ml of 40% HF in acetonitrile and then 10% pyridine was added. The mixture was stirred at room temperature, the reaction was monitored by TLC. After TLC shown that the reaction was completed, the mixture was quenched by the adding of 5% NaHCO₃ buffer and partitioning between water and ethyl acetate. The organic layer was separated, dried, and evaporated. The residue was purified by flash chromatography to give 13-(4′-phenyl-2′-t-butyloxy-oxazolidine-5′-carbonyloxy)-10-deacetylbaccatin III as white powder.

### 3. Deprotection

The product from step 2 was dissolved 200 ml of 5% HCl in EtOH and stirred at room temperature for 5 hours, the progress of the reaction was monitored by TLC. After TLC shown that the reaction was completed, the mixture was quenched by the adding saturated NaHCO₃ then partitioned between water and ethyl acetate. The organic layer was separated, dried with anhydrous Na₂SO₄, and evaporated. The residue was purified by flash chromatography to give the amino alcohol intermediate. To the amino alcohol intermediate dissolved in 200 ml of THF was added 1.5 equivalent of di-tert-butyldicarbonate followed by 1 equivalent of DMAP. The mixture was stirred at room temperature until the starting material was disappeared as monitored by TLC. The reaction was quenched by adding of 5% NaHCO₃ and partitioning between water and ethyl acetate. The combined organic phase was washed with brine and dried by anhydrous Na₂SO₄, then concentrated under vacuum to afford crude docetaxel which was purified through flash column chromatography to obtained docetaxel as white powder which identified by ¹H-NMR and HPLC through comparison with an authentic sample.

### Example 7

### Process for converting 9-Dihydro-13-acetylbaccatin III to Paclitaxel

Paclitaxel can be made according the methods disclosed in Example 5 by using 7-DMPS-9-dihydro-10-ketobaccatin III (Compound 5) and 3-N-Bz-4-phenyl-(4S,5R)-2,2-dioxo-1,2,3-oxathiazolidine carboxylic acid (side chain formula 11′).

### Example 8

### Process for converting 9-Dihydro-13-acetylbaccatin III to Docetaxel

100 Grams of 9-dihydro-13-acetylbaccatin III was dissolved in 1 litre of acetonitrile, after stirred for 5 minutes then one mole equivalent of sodium methoxide was added, the mixture was stirred at room temperature for 5 hours or until the reaction was completed (monitored by TLC). After normal work up the organic phase was collected and concentrated under vacuum, the white powder like product was identified by H-NMR as 10-deacetyl-9-dihydro-13-acetylbaccatin III (yield: 96%).

### Step 2: Protection of 7,10-Hydroxyl Group

100 Grams of 10-deacetyl-9-dihydro-13-acetylbaccatin III was dissolved in 700 ml of DMF, 4 mole of equivalent imidazole was added, the mixture was stirred at 0° C. for 10 minutes, then 10 mole equivalent of dimethylphenylsilyl chloride was added dropwise. The mixture was stirred for 1 hour then the temperature was warmed to about 20° C. and kept overnight at this temperature. The process was monitored by TLC, after work up the crude products were crystallized from acetone/hexanes mixed solvents. 102 Grams of crystal like product was obtained as white powder and identified as 7,10-di-DMPS-9-dihydro-10-deacetylbaccatin III. The final material can be used for next step reaction without pre-separation and purification through flash column chromatography.

### Step 3: Deacetylation at 13-Position

The product obtained from step 2 was dissolved into 3000 ml of 15% N₂H₄ (hydrazine monohydrate) in EtOH, the mixture was stirred at room temperature under nitrogen for 5 hours. After the reaction was shown to be completed by TLC analysis, the reaction was quenched by adding of brine and ethyl acetate. The organic phase was collected and evaporated to dryness under vacuum. The product 7,10-diDMPS-9-dihydro-10-deacetylbaccatin III was obtained as white solid and identified by ¹H-NMR (85 g).

### Step 4: Attaching Docetaxel Side Chain

The product obtained from step 3 was dissolved in 500 ml of methylbenzene and stirred at room temperature under nitrogen, 3 equivalent of docetaxel side chain precursors were then added. The mixture was stirred for 10 minutes before 4 mole equivalents DCC and 0.5 mole equivalent of DMAP were added. Then the temperature was raised to 70° C. and the mixture was kept stirred at this temperature for about 2 hours or until the reaction was completed. Work up as normal, the protected docetaxel derivative was obtained as white solid. (88 g)

### Step 5: Oxidation

The product from step 4 was dissolved in 1 liter of acetonitrile and stirred at room temperature (25° C.) until the solid completely dissolved. 4 Mole equivalent NMO and 0.05% (mole equivalent) of TPAP, and 30 grams of 4A molecular shiver were added. The mixture was stirred at room temperature overnight. After the reaction was completed which was stopped by adding water. The product was extracted with dichloromethane. The dichloromethane phase was then concentrated to dryness under vacuum, the product was purified through flash column chromatography. The material obtained as white solid (80 g).

### Step 6: Deprotection

The products from step 5 was dissolved 10% HF in ethanol, some pyridine was added and stirred at room temperature for 4 hours and monitored by TLC. After TLC shown that the reaction was completed, the mixture was quenched by the adding of 5% NaHCO₃ solution and partitioning between water and ethyl acetate. The organic layer was separated, dried, and evaporated. The residue was purified by flash chromatography to give docetaxel as white powder which identified by H-NMR and HPLC through comparison with an authentic sample.

### Example 9

### Reaction Scheme for Preparing Docetaxel and Paclitaxel

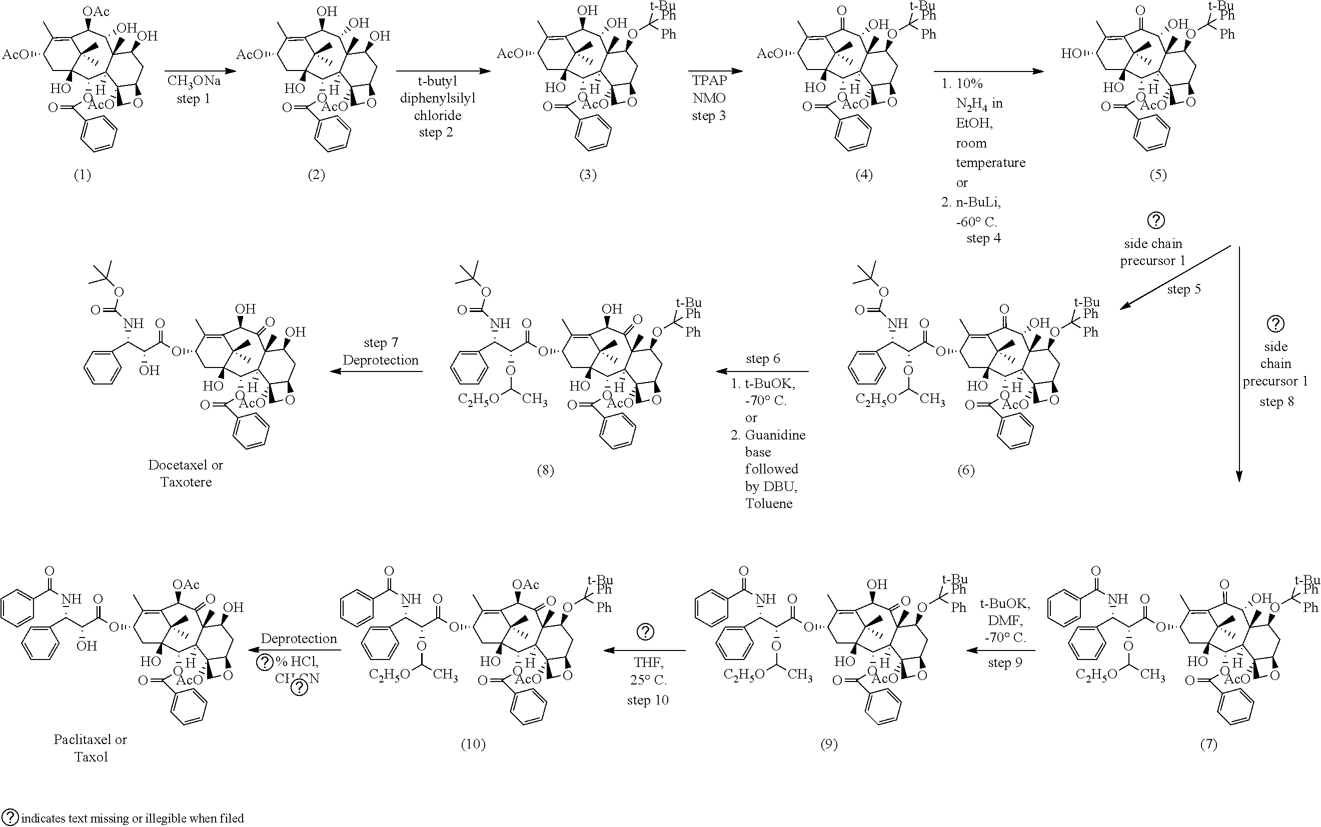

### Example 10

### Reaction Scheme for Preparing Docetaxel and Paclitaxel

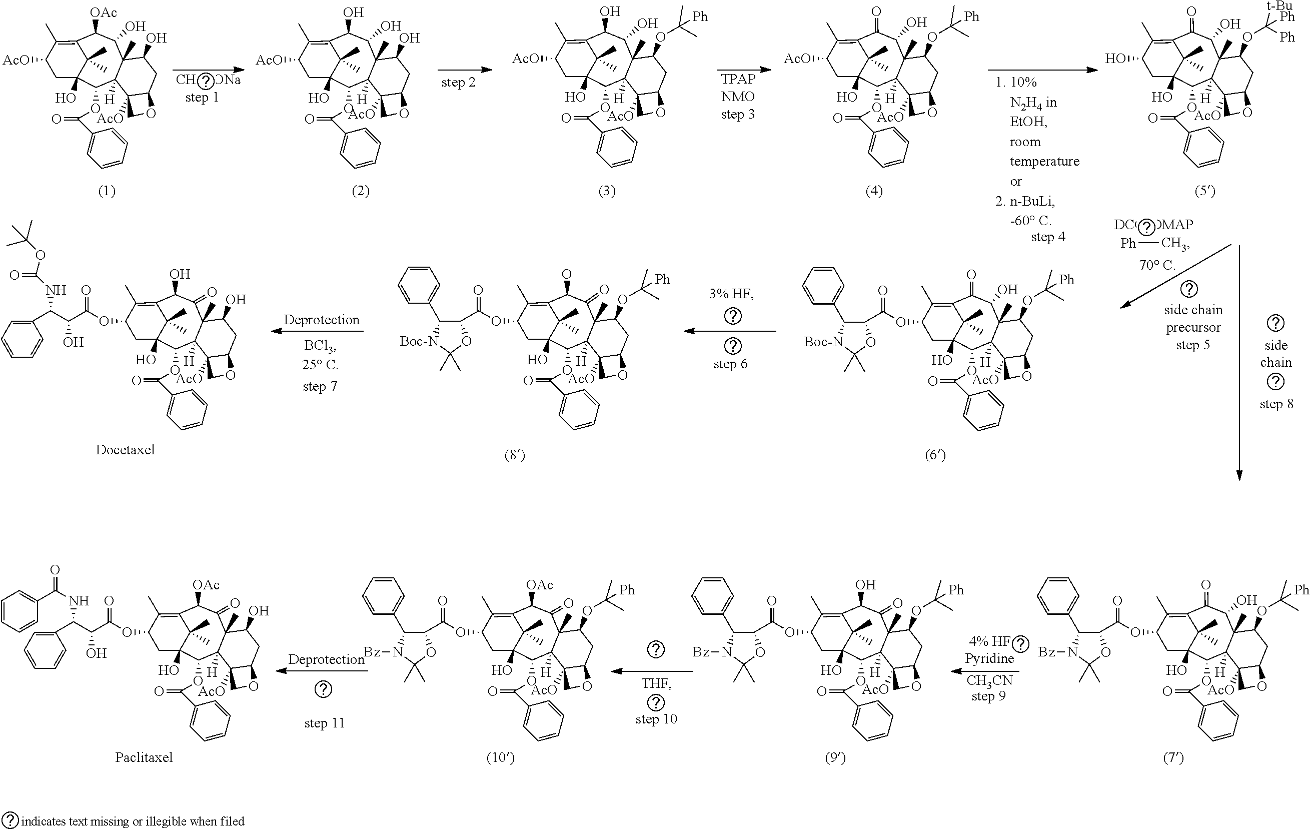

### Example 11

### Reaction Scheme for Preparing Docetaxel and Paclitaxel

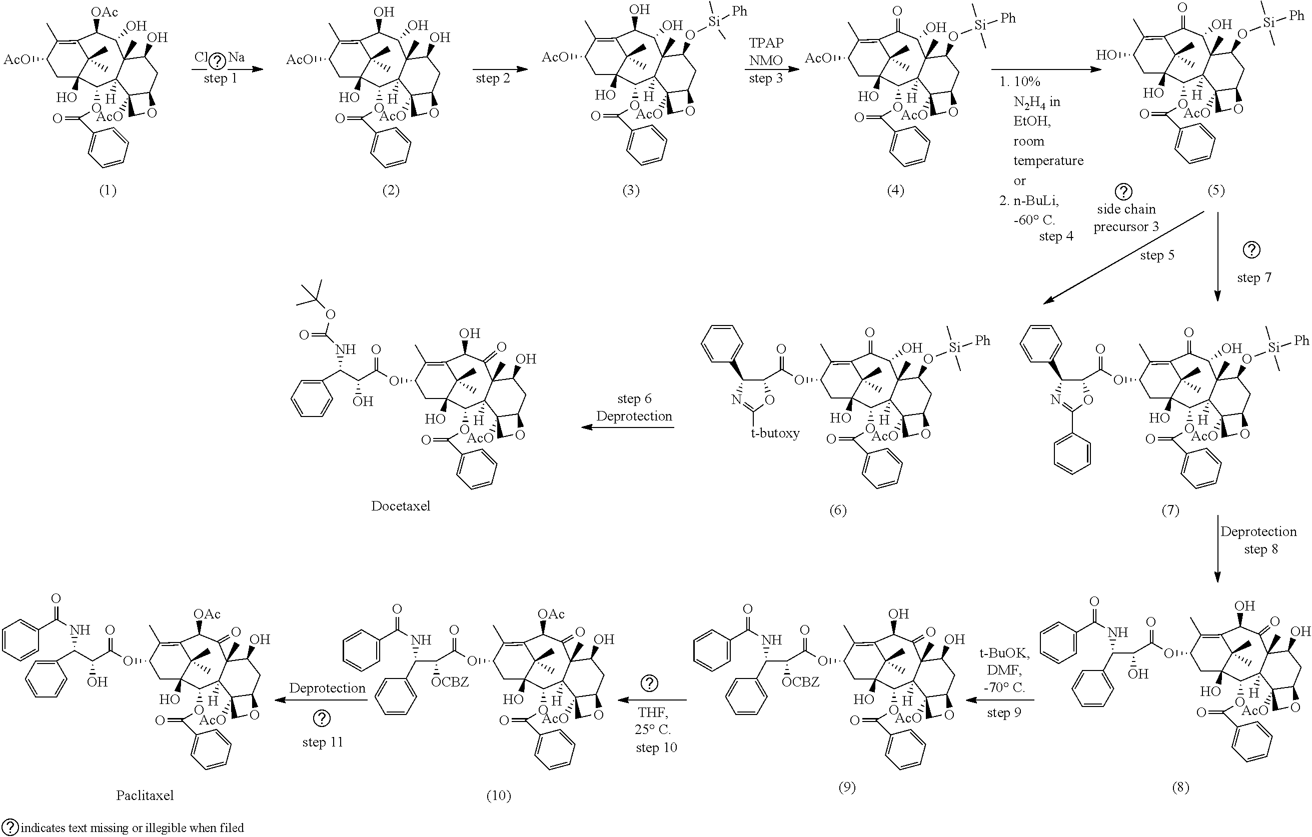

### Example 12

### Reaction Scheme for Preparing Docetaxel and Paclitaxel

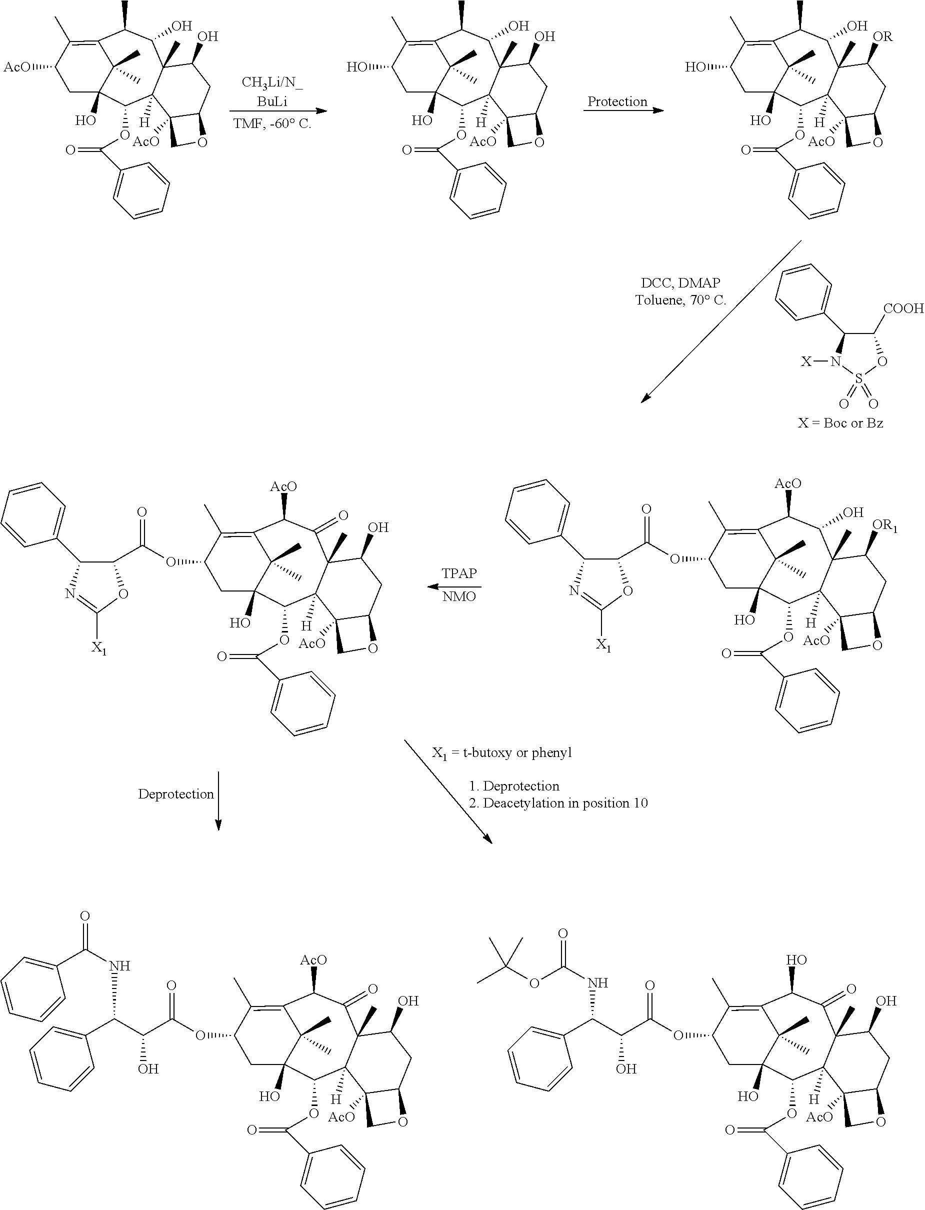

What is claimed is:

1. A compound of formula (2), (3), (4), (5), (6), (6′), (8), (8′), (10), (11), (12), (13) and (14): 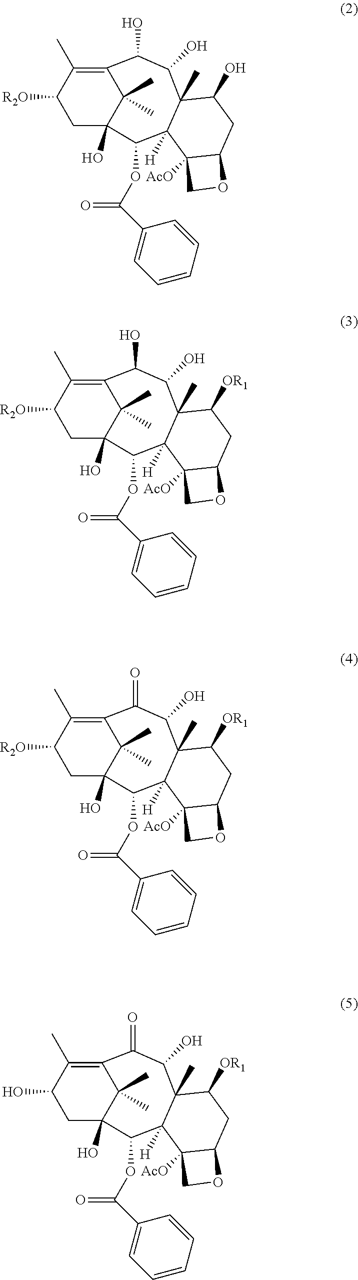 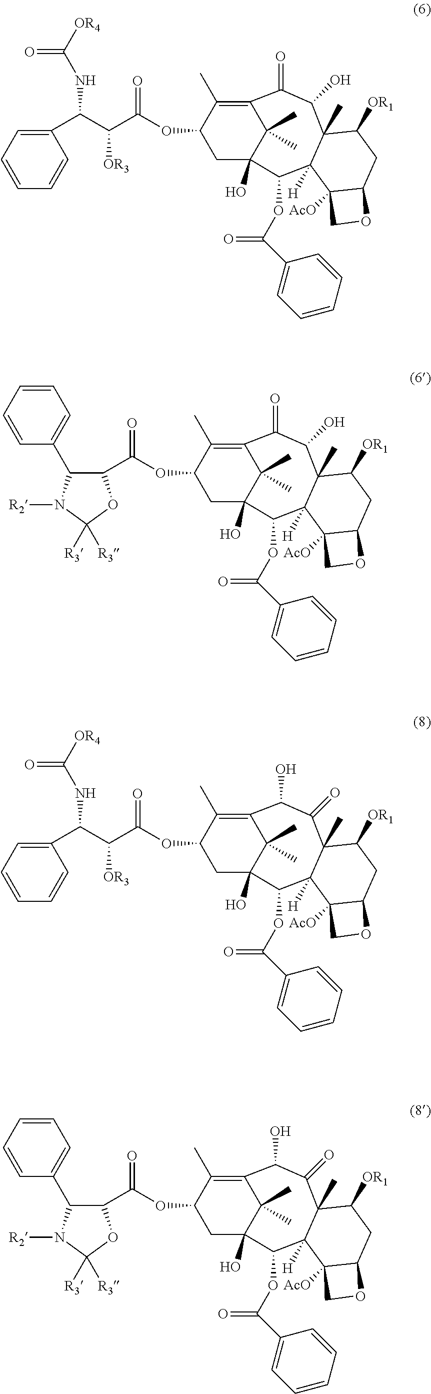 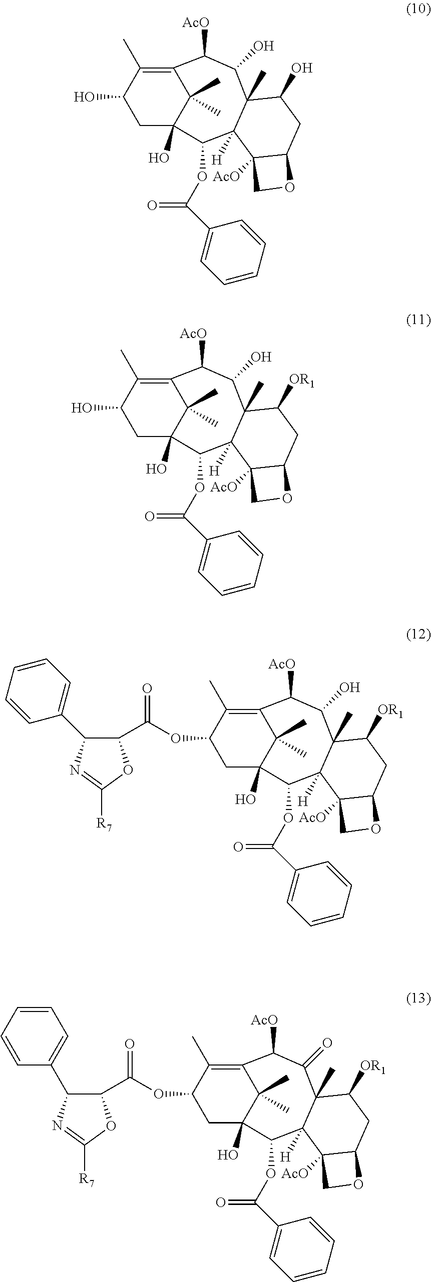 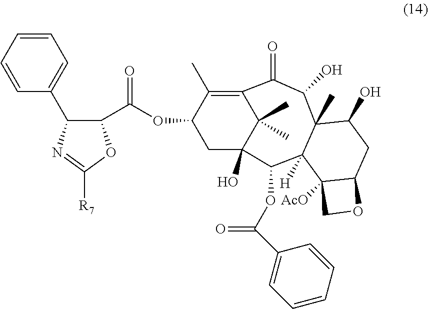
wherein in formula (3), (4), (5), (6), (6′), (8), (8′), (11), (12) and (13), R₁ is a hydrogen atom of a suitable hydroxyl-protecting group;
wherein in formula (2), (3) and (4), R₂ is a hydrogen atom or a suitable hydroxyl-protecting group;
wherein in formula (6′) and (8′), R₂′ is a suitable amino-protecting group and R₃′ and R₃″, identical or different, are a hydrogen atom or a methyl group; or R₂′ and one of R₃′ and R₃″ form together a π-bond and the other of R₃′ and R₃″ is a t-utoxy group or a phenyl group;
wherein in formula (6) and (8), R₃ is a hydrogen atom or a suitable hydroxyl-protecting group;
wherein in formula (6) and (8), R₄ is a hydrogen atom, a linear C₁-C₂₀ alkyl, a branched C₃-C₂₀ alkyl group, a C₁-C₂₀ acyl group, a C₁-C₂₀ halogenated acyl group, a C₃-C₁₂ cycloalkyl, a C₁-C₁₂ heterocyclyl, a C₂-C₂₀ alkenyl, a C₂-C₂₀ alkynyl, a C₆-C₁₂ aryl, a C₆-C₂₀ aralkyl, a C₁-C₂₀ alkyloxy C₆-C₂₀ alkylaryl, a C₁-C₁₂ heteroaryl, a C₂-C₂₀ alkylheterocyclyl or a C₂-C₂₀ alkylheteroaryl,
said alkyl, cycloalkyl, heterocyclyl, alkenyl, alkynyl, aryl, aralkyl, alkylaryl, heteroaryl, alkylheterocyclyl and alkylheteroaryl being unsubstituted or substituted with at least one substituent, each of said substituent(s) being selected from the group consisting of F, Cl, Br, I, OH, SH, NH₂, NO₂, CN, CF₃, —SH, —OCH₂Ph, —OPh, —SCH₃, —SPh, —SCH₂Ph,
—COOH, —COOR₆ (in which R₆ is a C₁-C₆ alkyl), linear C₁-C₂₀ alkyl, branched C₃-C₂₀ alkyl, C₆-C₁₂ aryl, C₂-C₂₀ alkenyl, C₁-C₂₀ alkoxy, C₂-C₂₀ alkynyl, C₆-C₂₀ aralkyl, C₆-C₁₂ aryl, C₃-C₈ cycloalkyl, C₁-C₂₀ aminoalkyl, C₆-C₁₂ aminoaryl, C₁-C₁₂ aminoheteroaryl, C₁-C₂₀ hydroxyalkyl, C₆-C₁₂ hydroxyaryl, hydroxyheteroaryl, C₁-C₁₂ heterocyclyl, C₁-C₁₂ heteroaryl, C₂-C₂₀ alkylheterocyclyl and C₂-C₂₀ alkylheteroaryl;
wherein in formula (12) and (13), R₇ is C₂-C₁₀ alkynyl, C₁-C₁₀ alkyl, C₂-C₁₀ alkenyl, C₁-C₁₀ alkoxy, C₆-C₁₂ aryl or C₅-C₁₂ heteroaryl, preferably a t-butoxy group or a phenyl group.

2. A compound of formula (2) and (3): 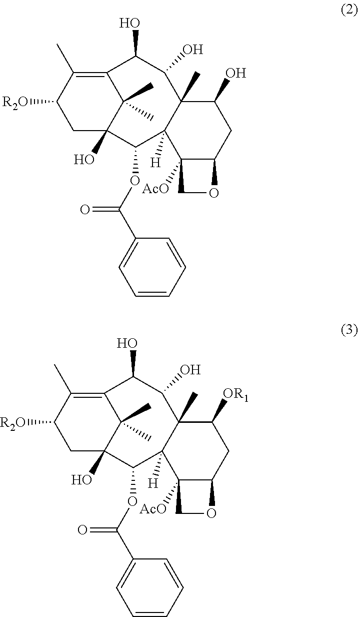
wherein in formula (3) R₁ is a hydrogen atom of a suitable hydroxyl-protecting group; and
wherein R₂ is a hydrogen atom or a suitable hydroxyl-protecting group.

3. A compound of formula (4) or (5): 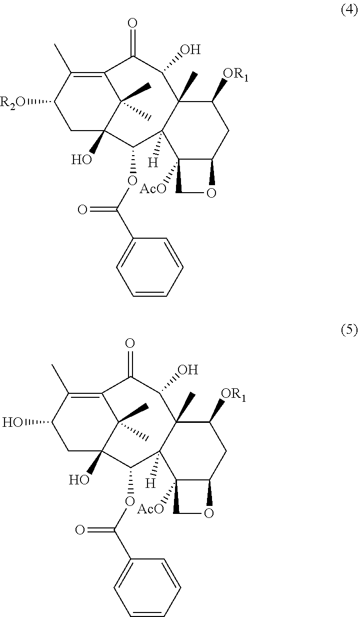
wherein R₁ is a hydrogen atom of a suitable hydroxyl-protecting group; and
wherein in formula (4) R₂ is a hydrogen atom or a suitable hydroxyl-protecting group.

4. A compound of formula (6) or (6′): 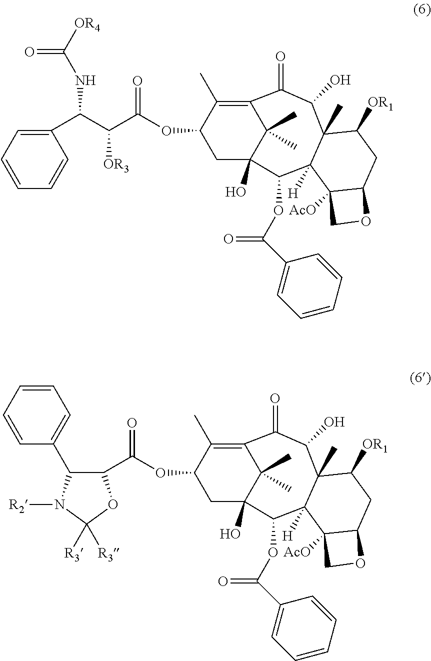
wherein R₁ is a hydrogen atom of a suitable hydroxyl-protecting group;
wherein in formula (6), R₄ is a hydrogen atom, a linear C₁-C₂₀ alkyl, a branched C₃-C₂₀ alkyl group, a C₁-C₂₀ acyl group, a C₁-C₂₀ halogenated acyl group, a C₃-C₁₂ cycloalkyl, a C₁-C₁₂ heterocyclyl, a C₂-C₂₀ alkenyl, a C₂-C₂₀ alkynyl, a C₆-C₁₂ aryl, a C₆-C₂₀ aralkyl, a C₁-C₂₀ alkyloxy C₆-C₂₀ alkylaryl, a C₁-C₁₂ heteroaryl, a C₂-C₂₀ alkylheterocyclyl or a C₂-C₂₀ alkylheteroaryl,
said alkyl, cycloalkyl, heterocyclyl, alkenyl, alkynyl, aryl, aralkyl, alkylaryl, heteroaryl, alkylheterocyclyl and alkylheteroaryl being unsubstituted or substituted with at least one substituent, each of said substituent(s) being selected from the group consisting of F, Cl, Br, I, OH, SH, NH₂, NO₂, CN, CF₃, —SH, —OCH₂Ph, —OPh, —SCH₃, —SPh, —SCH₂Ph, —COOH, —COOR₆ (in which R₆ is a C₁-C₆ alkyl), linear C₁-C₂₀ alkyl, branched C₃-C₂₀ alkyl, C₆-C₁₂ aryl, C₂-C₂₀ alkenyl, C₁-C₂₀ alkoxy, C₂-C₂₀ alkynyl, C₆-C₂₀ aralkyl, C₆-C₁₂ aryl, C₃-C₈ cycloalkyl, C₁-C₂₀ aminoalkyl, C₆-C₁₂ aminoaryl, C₁-C₁₂ aminoheteroaryl, C₁-C₂₀ hydroxyalkyl, C₆-C₁₂ hydroxyaryl, C₁-C₁₂ hydroxyheteroaryl, C₁-C₁₂ heterocyclyl, C₁-C₁₂ heteroaryl, C₂-C₂₀ alkylheterocyclyl and C₂-C₂₀ alkylheteroaryl;
wherein in formula (6), R₃ is a hydrogen atom or a hydroxyl-protecting group; and
wherein in formula (6′), R₂′ is a suitable amino-protecting group and R₃′ and R₃″, identical or different, are a hydrogen atom or a methyl group; or R₂′ and one of R₃′ and R₃″ form together a π-bond and the other of R₃′ and R₃″ is a t-butoxy group or a phenyl group.

5. Compound of formula (8) or (8′): 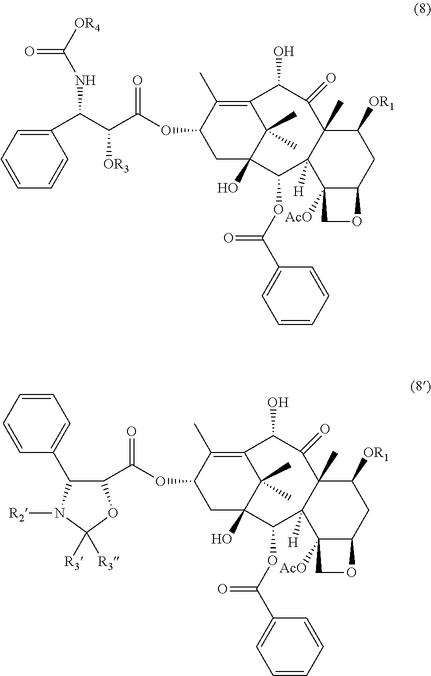
wherein R₁ is a hydrogen atom of a suitable hydroxyl-protecting group;
wherein in formula (8′) R₂′ is a suitable amino-protecting group and R₃′ and R₃″, identical or different, are a hydrogen atom or a methyl group; or R₂′ and one of R₃′ and R₃″ form together a π-bond and the other of R₃′ and R₃″ is a t-butoxy group or a phenyl group;
wherein in formula (8) R₃ is a hydrogen atom or a suitable hydroxyl-protecting group; and
wherein in formula (8) R₄ is a hydrogen atom, a linear C₁-C₂₀ alkyl, a branched C₃-C₂₀ alkyl group, a C₁-C₂₀ acyl group, a C₁-C₂₀ halogenated acyl group, a C₃-C₁₂ cycloalkyl, a C₁-C₁₂ heterocyclyl, a C₂-C₂₀ alkenyl, a C₂-C₂₀ alkynyl, a C₆-C₁₂ aryl, a C₆-C₂₀ aralkyl, a C₁-C₂₀ alkyloxy C₆-C₂₀ alkylaryl, a C₁-C₁₂ heteroaryl, a C₂-C₂₀ alkylheterocyclyl or a C₂-C₂₀ alkylheteroaryl,
said alkyl, cycloalkyl, heterocyclyl, alkenyl, alkynyl, aryl, aralkyl, alkylaryl, heteroaryl, alkylheterocyclyl and alkylheteroaryl being unsubstituted or substituted with at least one substituent, each of said substituent(s) being selected from the group consisting of F, Cl, Br, I, OH, SH, NH₂, NO₂, CN, CF₃, —SH, —OCH₂Ph, —OPh, —SCH₃, —SPh, —SCH₂Ph, —COOH, —COOR₆ (in which R₆ is a C₁-C₆ alkyl), linear C₁-C₂₀ alkyl, branched C₃-C₂₀ alkyl, C₆-C₁₂ aryl, C₂-C₂₀ alkenyl, C₁-C₂₀ alkoxy, C₂-C₂₀ alkynyl, C₆-C₂₀ aralkyl, C₆-C₁₂ aryl, C₃-C₈ cycloalkyl, C₁-C₂₀ aminoalkyl, C₆-C₁₂ aminoaryl, C₁-C₁₂ aminoheteroaryl, C₁-C₂₀ hydroxyalkyl, C₆-C₁₂ hydroxyaryl, C₁-C₁₂ hydroxyheteroaryl, C₁-C₁₂ heterocyclyl, C₁-C₁₂ heteroaryl, C₂-C₂₀ alkylheterocyclyl and C₂-C₂₀ alkylheteroaryl.

6. A compound of formula (10):

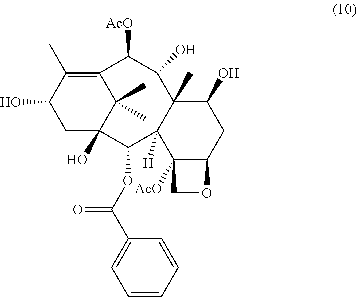

7. A compound of formula (11): 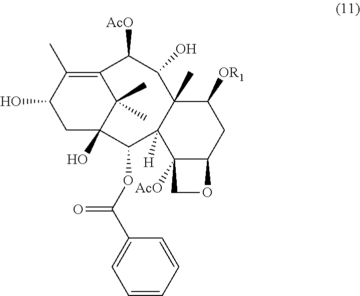
wherein R₁ is a hydrogen atom or a suitable hydroxyl-protecting group.

8. A compound of formula (12) 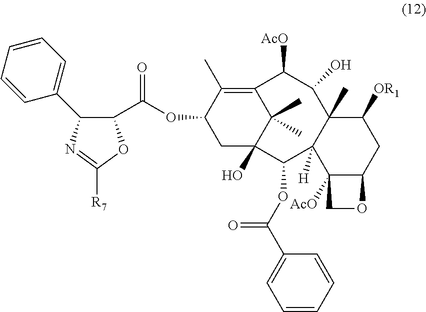
wherein R₁ is a hydrogen atom or a suitable hydroxyl-protecting group; and
wherein R₇ is C₂-C₁₀ alkynyl, C₁-C₁₀ alkyl, C₂-C₁₀ alkenyl, C₁-C₁₀ alkoxy, C₆-C₁₂ aryl or C₅-C₁₂ heteroaryl, preferably a t-butoxy group or a phenyl group.

9. A compound of formula (13): 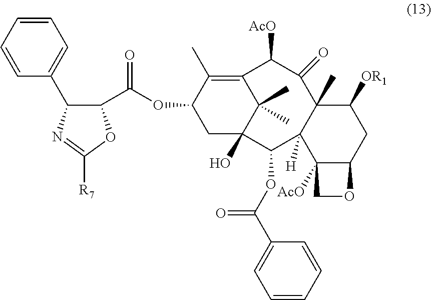
wherein R₁ is a hydrogen atom or a suitable hydroxyl-protecting group; and
wherein R₇ is C₂-C₁₀ alkynyl, C₁-C₁₀ alkyl, C₂-C₁₀ alkenyl, C₁-C₁₀ alkoxy, C₆-C₁₂ aryl or C₅-C₁₂ heteroaryl, preferably a t-butoxy group or a phenyl group.

10. A compound of formula (14): 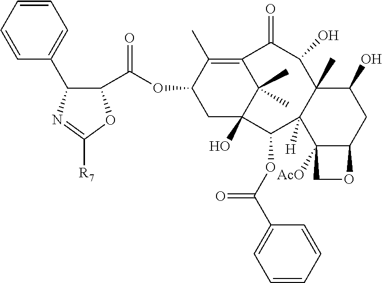
wherein R₇ is C₂-C₁₀ alkynyl, C₁-C₁₀ alkyl, C₂-C₁₀ alkenyl, C₁-C₁₀ alkoxy, C₆-C₁₂ aryl or C₅-C₁₂ heteroaryl.

11. The compound of anyone of claims 1 to 5 and 7 to 9, wherein said suitable hydroxyl-protecting group is selected from the group consisting of C₁-C₂₅ ethers, C₁-C₂₅ substituted methyl ethers, C₁-C₂₅ substituted ethyl ethers, C₁-C₂₅ acyl groups, C₁-C₂₅ halogenated acyl groups, C₁-C₂₅ substituted benzyl ethers, C₁-C₂₅ silyl ethers, C₁-C₂₅ esters, C₁-C₂₅ carbonates and C₁-C₂₅ sulfonates.

12. The compound of claim 11, wherein said suitable hydroxyl-protecting group is selected from the group consisting of methyl, methoxymethyl, benzyloxymethyl, tetrahydropyranyl, tetrahydrofuranyl, 2-(trimethylsilyl)ethoxymethyl, dioxanyl, 1-ethoxyethyl, 1-(2-chloroethoxy)ethyl, 2,2,2-trichloroethyl, t-butyl, allyl, propargyl, benzyl, ρ-methoxybenzyl, diphenylmethyl, triphenylmethyl, trimethylsilyl, triethylsilyl, triisopropylsilyl, dimethylisopropylsilyl, diethylisopropylsilyl, dimethylthexylsilyl, t-butyldimethylsilyl, t-butyldiphenylsilyl, tribenzylsilyl, triphenylsilyl, diphenylmethylsilyl, phenyldimethylsilyl, benzylformate, methylcarbonyl, ethylcarbonyl, methoxymethyl arbonyl, trichloroethoxycarbonyl, benzylcarbonyl, benzyloxycarbonyl allylsulfonyl, methanesulfonyl and p-toluenesulfonyl.

13. The compound of any one of claims 1 to 5 and 7 to 9, wherein R₁ is a hydroxyl-protecting group of formula: 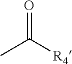
wherein R₄′ forms with the carbonyl a C₁-C₂₀ acyl group or a C₁-C₂₀ halogenated acyl group.

14. The compound of any one of claims 1 to 5 and 7 to 9, wherein R₁ is t-butyldiphenyl silyl, diphenylmethylsilyl or phenyldimethylsilyl.

15. The compound of any one of claims 1, 4 and 5, wherein R₁ is phenyldimethylsilyl, R₂′ and R₃′ form together a π-bond and R₃″ is t-butoxy.

16. The compound of any one of claims 1, 4 and 5, wherein R₁ is phenyldimethylsilyl, R₂′ is Boc, R₃′ and R₃″ are methyl.

17. The compound of any one of claims 1, 4 and 5, wherein R₁ is phenyldimethylsilyl, R₂′ is benzyl, R₃′ and R₃″ are methyl.

18. The compound of any one of claims 1, 4 and 5, wherein R₁ is phenyldimethylsilyl, R₂ is absent, R₃ is a hydrogen atom and R_{3′} is absent.

19. The compound of any one of claims 1, 4 and 5, wherein R₁ is phenyldimethylsilyl, R₂ is absent, R₃ is ethoxyethyl and R_{3′} is absent.

20. The compound of any one of claims 1, 4 and 5, wherein R₁ is phenyldimethylsilyl, R₂′ and R₃′ form together a π-bond and R₃″ is phenyl.

21. The compound of any one of claims 1 to 3, wherein R₁ is a hydrogen atom, and R₂ is an acetyl group.

22. The compound of any one of claims 1 to 3, wherein R₂ is acetyl.

23. The compound of any one of claims 1, 4, 5 and 22, wherein R₃ is ethoxyethyl.

24. The compound of any one of claims 1, 4, 5, 22 and 23, wherein R₄ is a C₁-C₆ alkyl, phenyl, t-butyloxyl, a C₂-C₆ alkenyl, tetrahydrofuranyl or tetrahydropyranyl.

25. The compound of any one of claims 1, 4, 5, 22, 23 and 24, wherein R₄ is a t-butyloxyl.

26. Compound according to any one of claims 1, 4 and 5, wherein R₁ is t-butyldiphenyl silyl, diphenylmethylsilyl or phenyldimethylsilyl, R₃ is ethoxyethyl, R₄ is t-butyloxyl.

27. Process for the preparation of a compound of formula (4): 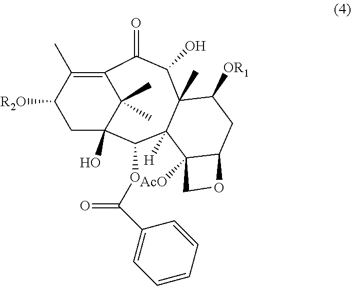
wherein R₁ is a hydrogen atom of a suitable hydroxyl-protecting group; and
wherein R₂ is a hydrogen atom or a suitable hydroxyl-protecting group;
said process comprising the step of oxidating a compound of formula (3): 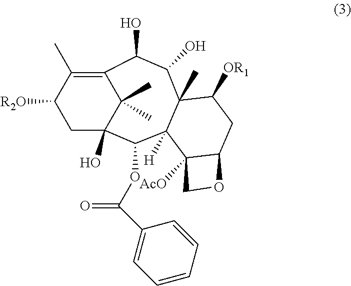
wherein R₁ and R₂ are as defined hereinbefore.

28. A process according to claim 27, wherein R₁ is t-butyldiphenyl silyl, diphenylmethylsilyl or phenyldimethylsilyl and R₂ is acetyl.

29. Process for the preparation of a compound of formula (5): 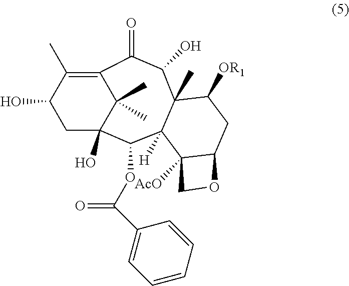
wherein R₁ is a hydrogen atom of a suitable hydroxyl-protecting group,
said process comprising the step of removing the protecting group in 13 position of a compound of formula (4): 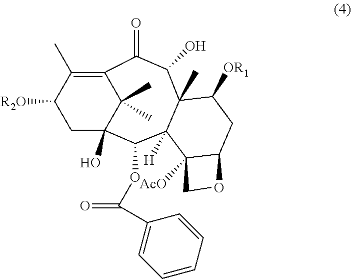
wherein R₁ is as defined hereinabove and R₂ is a suitable hydroxyl-protecting group.

30. Process according to claim 29, wherein R₂ is t-butyldiphenyl silyl, diphenylmethylsilyl or phenyldimethylsilyl and the step for removing the protecting group in the 13 position is carried out with n-butyl lithium at −60° C.

31. Process for the preparation of a compound of formula (6): 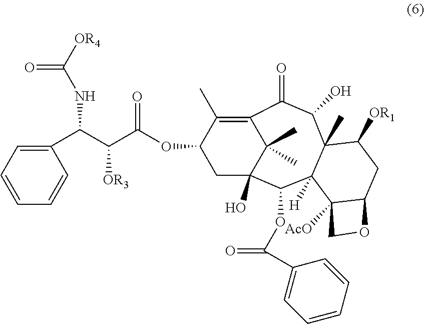
wherein R₁ is a hydrogen atom of a suitable hydroxyl-protecting group;
wherein R₃ is a hydrogen atom or a suitable hydroxyl-protecting group; and
wherein R₄ is a hydrogen atom, a linear C₁-C₂₀ alkyl, a branched C₃-C₂₀ alkyl group, a C₁-C₂₀ acyl group, a C₁-C₂₀ halogenated acyl group, a C₃-C₁₂ cycloalkyl, a C₁-C₁₂ heterocyclyl, a C₂-C₂₀ alkenyl, a C₂-C₂₀ alkynyl, a C₆-C₁₂ aryl, a C₆-C₂₀ aralkyl, a C₁-C₂₀ alkyloxy C₆-C₂₀ alkylaryl, a C₁-C₁₂ heteroaryl, a C₂-C₂₀ alkylheterocyclyl or a C₂-C₂₀ alkylheteroaryl,
said alkyl, cycloalkyl, heterocyclyl, alkenyl, alkynyl, aryl, aralkyl, alkylaryl, heteroaryl, alkylheterocyclyl and alkylheteroaryl being unsubstituted or substituted with at least one substituent, each of said substituent(s) being selected from the group consisting of F, Cl, Br, I, OH, SH, NH₂, NO₂, CN, CF₃, —SH, —OCH₂Ph, —OPh, —SCH₃, —SPh, —SCH₂Ph, —COOH, —COOR₆ (in which R₆ is a C₁-C₆ alkyl), linear C₁-C₂₀ alkyl, branched C₃-C₂₀ alkyl, C₆-C₁₂ aryl, C₂-C₂₀ alkenyl, C₁-C₂₀ alkoxy, C₂-C₂₀ alkynyl, C₆-C₂₀ aralkyl, C₆-C₁₂ aryl, C₃-C₈ cycloalkyl, C₁-C₂₀ aminoalkyl, C₆-C₁₂ aminoaryl, C₁-C₁₂ aminoheteroaryl, C₁-C₂₀ hydroxyalkyl, C₆-C₁₂ hydroxyaryl, C₁-C₁₂ hydroxyheteroaryl, C₁-C₁₂ heterocyclyl, C₁-C₁₂ heteroaryl, C₂-C₂₀ alkylheterocyclyl and C₂-C₂₀ alkylheteroaryl;
said process comprising the step of reacting a precursor of side chain of formula: 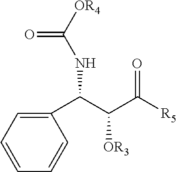
wherein R₃ and R₄ are as defined hereinabove, and R₅ is a radical suitable to add said side chain in the 13 position of the compound of formula (5): 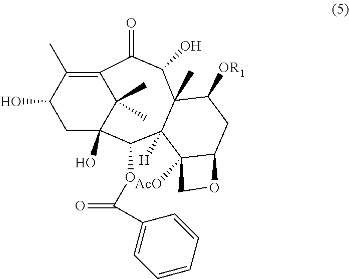
wherein R₁ is as defined hereinabove, to form said compound of formula (6).

32. Process according to claim 31, wherein R₁ is t-butyldiphenyl silyl, diphenylmethylsilyl or phenyldimethylsilyl, R₃ is ethoxyethyl, R₄ is t-butyloxyl, and R₅ is a hydroxyl group.

33. Process for the preparation of compounds of formula (6′): 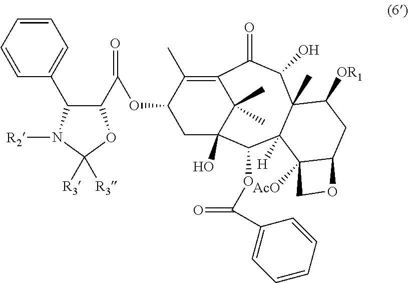
wherein R₁ is a hydrogen atom of a suitable hydroxyl-protecting group; and
wherein R₂′ is a suitable amino-protecting group and R₃′ and R₃″, identical or different, are a hydrogen atom or a methyl group; or R₂′ and one of R₃′ and R₃″ form together a π-bond and the other of R₃′ and R₃″ is a t-butoxy group or a phenyl group, said process comprising the step of reacting a precursor of side chain of formula: 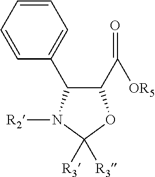
wherein R₂′, R₃′ and R₃″ are as defined hereinabove, and R₅ is a radical suitable to add said side chain in the 13 position of the compound of formula (5): 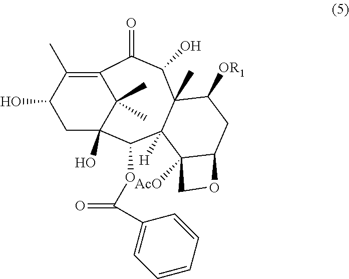
wherein R₁ is as defined hereinabove.

34. Process for preparing a compound of formula (8): 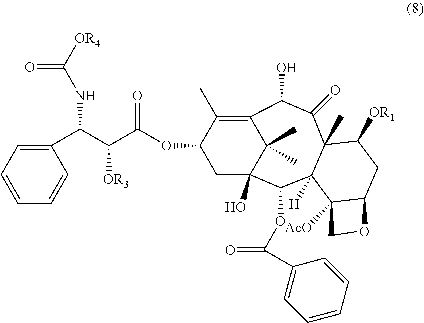
wherein R₁ is a hydrogen atom of a suitable hydroxyl-protecting group;
wherein R₂′ is a suitable amino-protecting group and R₃′ and R₃″, identical or different, are a hydrogen atom or a methyl group; or R₂′ and one of R₃′ and R₃″ form together a n-bond and the other of R₃′ and R₃″ is a hydrogen atom or a methyl group;
wherein R₃ is a hydrogen atom or a suitable hydroxyl-protecting group; and
wherein R₄ is a hydrogen atom, a linear C₁-C₂₀ alkyl, a branched C₃-C₂₀ alkyl group, a C₁-C₂₀ acyl group, a C₁-C₂₀ halogenated acyl group, a C₃-C₁₂ cycloalkyl, a C₁-C₁₂ heterocyclyl, a C₂-C₂₀ alkenyl, a C₂-C₂₀ alkynyl, a C₆-C₁₂ aryl, a C₆-C₂₀ aralkyl, a C₁-C₂₀ alkyloxy C₆-C₂₀ alkylaryl, a C₁-C₁₂ heteroaryl, a C₂-C₂₀ alkylheterocyclyl or a C₂-C₂₀ alkylheteroaryl,
said alkyl, cycloalkyl, heterocyclyl, alkenyl, alkynyl, aryl, aralkyl, alkylaryl, heteroaryl, alkylheterocyclyl and alkylheteroaryl being unsubstituted or substituted with at least one substituent, each of said substituent(s) being selected from the group consisting of F, Cl, Br, I, OH, SH, NH₂, NO₂, CN, CF₃, —SH, —OCH₂Ph, —OPh, —SCH₃, —SPh, —SCH₂Ph, —COOH, —COOR₆ (in which R₆ is a C₁-C₆ alkyl), linear C₁-C₂₀ alkyl, branched C₃-C₂₀ alkyl, C₆-C₁₂ aryl, C₂-C₂₀ alkenyl, C₁-C₂₀ alkoxy, C₂-C₂₀ alkynyl, C₆-C₂₀ aralkyl, C₆-C₁₂ aryl, C₃-C₈ cycloalkyl, C₁-C₂₀ aminoalkyl, C₆-C₁₂ aminoaryl, C₁-C₁₂ aminoheteroaryl, C₁-C₂₀ hydroxyalkyl, C₆-C₁₂ hydroxyaryl, C₁-C₁₂ hydroxyheteroaryl, C₁-C₁₂ heterocyclyl, C₁-C₁₂ heteroaryl, C₂-C₂₀ alkylheterocyclyl and C₂-C₂₀ alkylheteroaryl; comprising a step of intramolecular isomerization a compound of formula (6): 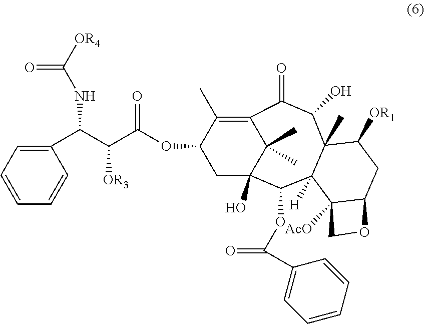
wherein R₁, R₃ and R₄ are as defined hereinabove.

35. Process according to claim 34, wherein R₁ is t-butyldiphenyl silyl, diphenylmethylsilyl or phenyldimethylsilyl, R₃ is ethoxyethyl, R₄ is t-butyloxyl.

36. Process according to claim 34 or 35, wherein the intramolecular isomerization is obtained by subjecting the compound of formula (6) to a guanidine base in methylene chloride.

37. process for the preparation of a compound of formula (8′): 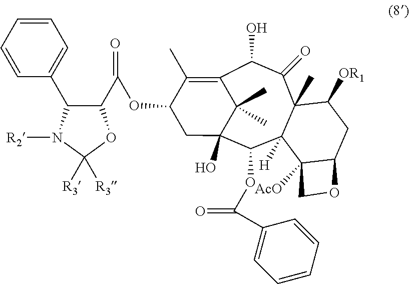
wherein R₁ is a hydrogen atom of a suitable hydroxyl-protecting group; and
wherein R₂′ is a suitable amino-protecting group and R₃′ and R₃″, identical or different, are a hydrogen atom or a methyl group; or R₂′ and one of R₃′ and R₃″ form together a π-bond and the other of R₃′ and R₃″ is a t-butoxy group or a phenyl group. This process advantageously comprises a step of intramolecular isomerization a compound of formula (6′): 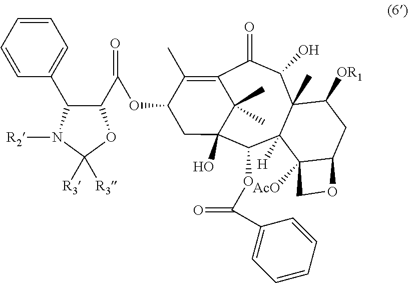
wherein R₁ is a hydrogen atom of a suitable hydroxyl-protecting group; and
wherein in formula (6′), R₂′ is a suitable amino-protecting group and R₃′ and R₃″, identical or different, are a hydrogen atom or a methyl group; or R₂′ and one of R₃′ and R₃″ form together a π-bond and the other of R₃′ and R₃″ is a t-butoxy group or a phenyl group.

38. Process for preparing docetaxel and derivative thereof, comprising a step of intramolecular isomerization a compound of formula (6): 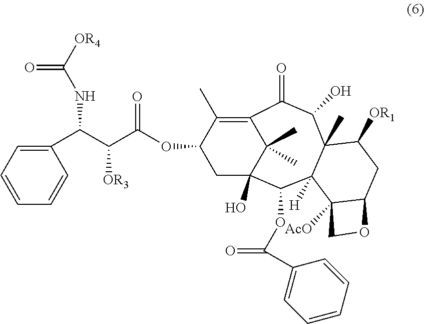
wherein R₁ is a hydrogen atom of a suitable hydroxyl-protecting group;
wherein R₃ is a hydrogen atom or a suitable hydroxyl-protecting group; and
wherein R₄ is a hydrogen atom, a linear C₁-C₂₀ alkyl, a branched C₃-C₂₀ alkyl group, a C₁-C₂₀ acyl group, a C₁-C₂₀ halogenated acyl group, a C₃-C₁₂ cycloalkyl, a C₁-C₁₂ heterocyclyl, a C₂-C₂₀ alkenyl, a C₂-C₂₀ alkynyl, a C₆-C₁₂ aryl, a C₆-C₂₀ aralkyl, a C₁-C₂₀ alkyloxy C₆-C₂₀ alkylaryl, a C₁-C₁₂ heteroaryl, a C₂-C₂₀ alkylheterocyclyl or a C₂-C₂₀ alkylheteroaryl,
said alkyl, cycloalkyl, heterocyclyl, alkenyl, alkynyl, aryl, aralkyl, alkylaryl, heteroaryl, alkylheterocyclyl and alkylheteroaryl being unsubstituted or substituted with at least one substituent, each of said substituent(s) being selected from the group consisting of F, Cl, Br, I, OH, SH, NH₂, NO₂, CN, CF₃, —SH, —OCH₂Ph, —OPh, —SCH₃, —SPh, —SCH₂Ph, —COOH, —COOR₆ (in which R₆ is a C₁-C₆ alkyl), linear C₁-C₂₀ alkyl, branched C₃-C₂₀ alkyl, C₆-C₁₂ aryl, C₂-C₂₀ alkenyl, C₁-C₂₀ alkoxy, C₂-C₂₀ alkynyl, C₆-C₂₀ aralkyl, C₆-C₁₂ aryl, C₃-C₈ cycloalkyl, C₁-C₂₀ aminoalkyl, C₆-C₁₂ aminoaryl, C₁-C₁₂ aminoheteroaryl, C₁-C₂₀ hydroxyalkyl, C₆-C₁₂ hydroxyaryl, C₁-C₁₂ hydroxyheteroaryl, C₁-C₁₂ heterocyclyl, C₁-C₁₂ heteroaryl, C₂-C₂₀ alkylheterocyclyl and C₂-C₂₀ alkylheteroaryl; by addition of at least one intramolecular isomerization agent to transform said compound of formula (4) into a compound of formula (7): 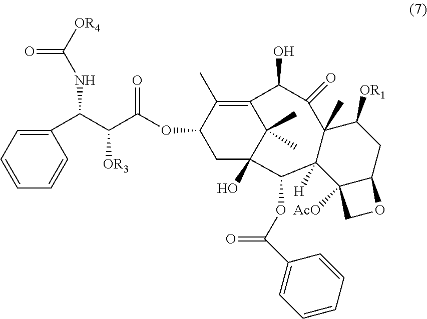
wherein R₁, R₃ and R₄ are as defined hereinabove, followed if necessary by a deprotection step removing eventual protective groups defined by R₁, R₃ and R₄.

39. Process according to claim 38, wherein R₁ is t-butyldiphenyl silyl, diphenylmethylsilyl or phenyldimethylsilyl; R₃ is ethoxyethyl; R₄ is t-butyloxyl.

40. Process according to claim 39, wherein the deprotection step is carried out in with HF.

41. Process according to claim 38, 39 or 40, wherein the intramolecular isomerization is obtained by subjecting the compound of formula (6) to a guanidine base in methylene chloride and then subjecting the product obtained to a 1,8-diazabicyclo[5,4,0]undec-7-ene in toluene.

42. Process for preparing a compound of formula (3): 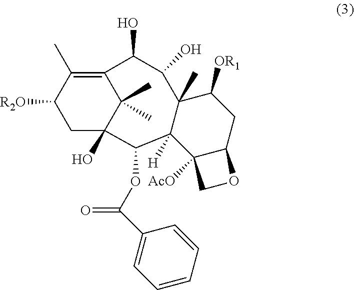
wherein R₁ is a hydrogen atom of a suitable hydroxyl-protecting group; and
wherein R₂ is a hydrogen atom or a suitable hydroxyl-protecting group,
said process comprising a step of reacting a compound of formula (2): 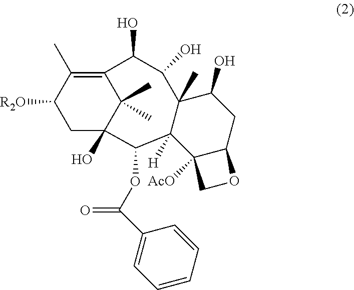
wherein R₂ is as defined hereinabove, in the presence of an agent suitable to protect the hydroxyl in the 7 position.

43. Process according to claim 40, wherein the agent suitable to protect the hydroxyl group in the 7 position is t-butyldiphenylsilyl chloride, t-butyldiphenyl silyl, diphenylmethylsilyl or phenyldimethylsilyl.

44. Process for preparing a compound of formula (2): 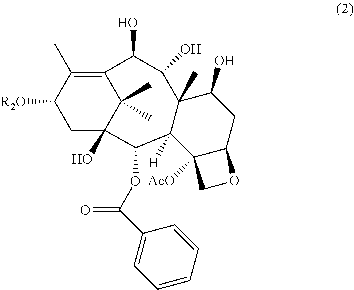
wherein R₂ is a hydrogen atom or a suitable hydroxyl-protecting group;
said process comprising the deacetylation of the acetyl group in position 10 of the 9-dihydro-13-acetylbaccatin III of formula (1): 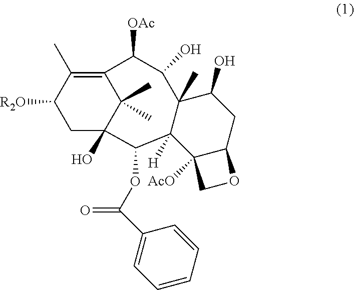
wherein R₂ is as defined hereinabove.

45. Process for the preparation of a compound of formula (10): 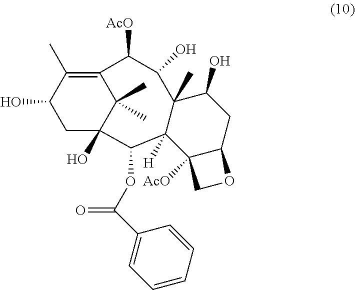
said process comprising the step of submitting a compound of formula (9): 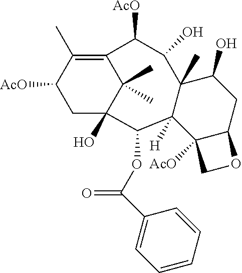
to CH₃Li/n.BuLi in THF at −60° C.

46. Process for the preparation of a compound of formula (11): 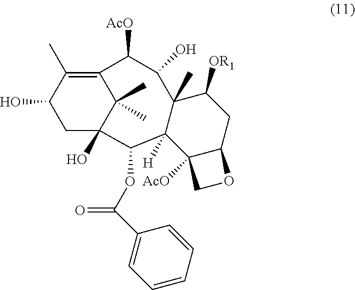
wherein R₁ is a hydrogen atom or a suitable hydroxyl-protecting group, said process comprising a step of protecting the hydroxyl group in position the 7 in of compound of formula (10): 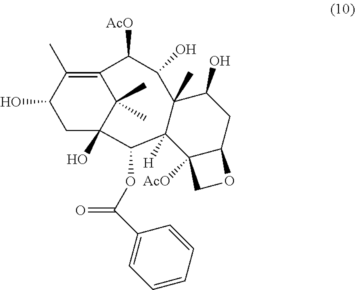
with a hydroxyl-protecting group.

47. Process according to claim 46, wherein said suitable protecting group for a hydroxyl group is selected from the group consisting of C₁-C₂₅ ethers, C₁-C₂₅ substituted methyl ethers, C₁-C₂₅ substituted ethyl ethers, C₁-C₂₅ acyl groups, C₁-C₂₅ halogenated acyl groups, C₁-C₂₅ substituted benzyl ethers, C₁-C₂₅ silyl ethers, C₁-C₂₅ esters, C₁-C₂₅ carbonates, and C₁-C₂₅ sulfonates.

48. Process according to claim 47, wherein said suitable protecting group for a hydroxyl group is selected from the group consisting of methyl, methoxymethyl, benzyloxymethyl, tetrahydropyranyl, tetrahydrofuranyl, 2-(trimethylsilyl)ethoxymethyl, dioxanyl, 1-ethoxyethyl, 1-(2-chloroethoxy)ethyl, 2,2,2-trichloroethyl, t-butyl, allyl, propargyl, benzyl, ρ-methoxybenzyl, diphenylmethyl, triphenylmethyl, trimethylsilyl, triethylsilyl, triisopropylsilyl, dimethylisopropylsilyl, diethylisopropylsilyl, dimethylthexylsilyl, t-butyldimethylsilyl, t-butyldiphenylsilyl, tribenzylsilyl, triphenylsilyl, diphenylmethylsilyl, phenyldimethylsilyl, benzylformate, methylcarbonyl, ethylcarbonyl, methoxymethyl arbonyl, trichloroethoxycarbonyl, benzylcarbonyl, benzyloxycarbonyl allylsulfonyl, methanesulfonyl, and p-toluenesulfonyl.

49. Process according to claim 46, wherein R₁ is a hydroxyl-protecting group of formula: 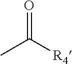
wherein R₄′ forms with the carbonyl a C₁-C₂₀ acyl group or a C₁-C₂₀ halogenated acyl group.

50. Process for the preparation of a compound of formula (12): 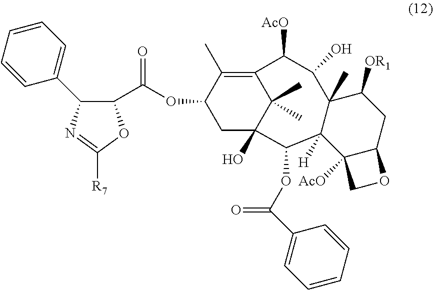
wherein R₁ is a hydrogen atom or a suitable hydroxyl-protecting group; and
wherein R₇ is C₂-C₁₀ alkynyl, C₁-C₁₀ alkyl, C₂-C₁₀ alkenyl, C₁-C₁₀ alkoxy, C₆-C₁₂ aryl or C₅-C₁₂ heteroaryl, preferably a t-butoxy group or a phenyl group, said process comprising the step of reacting a compound of formula (11): 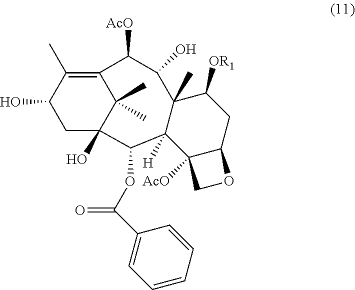
wherein R₁ is a hydrogen atom or a suitable protecting group for a hydroxyl group, with a compound of formula:
with a compound of formula: 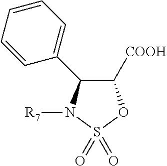
wherein X represents a radical of formula R₇—CO— where R₇ is C₂-C₁₀ alkynyl, C₁-C₁₀ alkyl, C₂-C₁₀ alkenyl, C₁-C₁₀ alkoxy, C₆-C₁₂ aryl or C₅-C₁₂ heteroaryl, preferably a t-butoxy group or a phenyl group, in the presence of DCG, DMAP and toluene at 70° C.

51. Process for the preparation of a compound of formula (13): 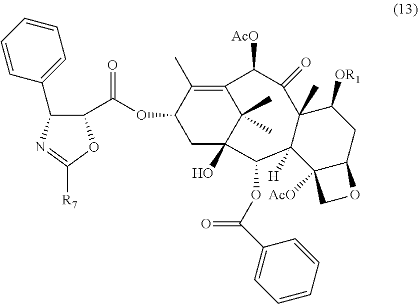
wherein R₁ is a hydrogen atom or a suitable hydroxyl-protecting group; and
wherein R₇ is C₂-C₁₀ alkynyl, C₁-C₁₀ alkyl, C₂-C₁₀ alkenyl, C₁-C₁₀ alkoxy, C₆-C₁₂ aryl or C₅-C₁₂ heteroaryl, preferably a t-butoxy group or a phenyl group, said process comprising a step submitting a compound of formula (12): 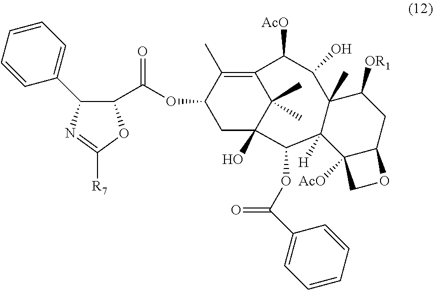
wherein R₁ and R₇ are as defined hereinabove, to the presence of TPAP and NMO.
